(19) United States
(12) Patent Application Publication
(10) Pub. No.: US 2011/0264098 A1
(43) Pub. Date: Oct. 27, 2011

(54) MINIMALLY INVASIVE SYSTEMS, DEVICES, AND SURGICAL METHODS FOR PERFORMING ARTHRODESIS IN THE SPINE

(76) Inventor: CHARLES S. COBBS, San Francisco, CA (US)

(21) Appl. No.: 13/034,150

(22) Filed: Feb. 24, 2011

Related U.S. Application Data
(60) Provisional application No. 61/338,982, filed on Feb. 26, 2010.

(51) Int. Cl. A61B 17/58

(52) U.S. Cl. 606/93

(57) ABSTRACT

Systems, devices, and methods achieve percutaneous fusion of the spine. The systems, devices, and methods percutaneously manipulate instrumentation to achieve posterior percutaneous transpedicular access to an interior of a first targeted vertebral body through a pedicle of the vertebra. The systems, devices, and methods percutaneously manipulate instrumentation through the achieved percutaneous transpedicular access, to achieve percutaneous cephalad trans-disc access to an interior of a second targeted vertebral body at a next adjacent superior level to the first targeted vertebral body. The systems, devices, and methods percutaneously manipulate instrumentation through the achieved percutaneous transpedicular access and percutaneous cephalad trans-disc access, to achieve percutaneous disc cavity creation comprising forming an enlarged cavity in the intervertebral disc space between the first and second targeted vertebral bodies. The systems, devices, and methods percutaneously manipulate instrumentation through the achieved percutaneous transpedicular access and percutaneous cephalad trans-disc access, to place a support structure in the enlarged cavity that achieves disc cavity support, into which a volume of a filling material is conveyed that, over time, hardens to promote fusion of the targeted first and second vertebral bodies.

### RELATED APPLICATION

This application claims the benefit of U.S. Provisional Patent Application Ser. No. 61/338,982, filed Feb. 26, 2010, and entitled “Minimally Invasive Systems, Devices, and Surgical Methods for Performing Arthrodesis in the Spine.”

### FIELD OF THE INVENTION

The invention generally relates to systems, devices, and surgical methods for the treatment of various types of spinal pathologies. More specifically, the invention is directed to systems, devices, and surgical methods for performing arthrodesis, or bone fusion, between vertebrae in the spine using minimally invasive instrumentation and techniques.

### BACKGROUND OF THE INVENTION

Back pain is a common complaint. Four out of five people in the United States will experience low back pain at least once during their lives. It is one of the most common reasons people go to the doctor or miss work. Back pain usually originates from the muscles, nerves, bones, joints or other structures in the spine.

The spine (see FIG. 1) is a complex interconnecting network of nerves, joints, muscles, tendons and ligaments, and all are capable of producing pain.

The spine is made up of small bones, called vertebrae (see FIGS. 2A and 2B). The vertebrae protect and support the spinal cord. They also bear the majority of the weight put upon the spine. As can be best seen in FIG. 2B, vertebrae, like all bones, have an outer shell called cortical bone (the vertebral body) that is hard and strong. The inside is made of a soft, spongy type of bone, called cancellous bone. The bony plates or processes of the vertebrae that extend rearward and laterally from the vertebral body provide a bony protection for the spinal cord and emerging nerves.

Between each vertebra is a soft, gel-like “cushion,” called an intervertebral disc. These flat, round cushions act like shock absorbers by helping absorb pressure and keep the bones from rubbing against each other. The intervertebral disc also binds adjacent vertebrae together. The intervertebral discs are a type of joint in the spine. Intervertebral disc joints can bend and rotate a bit but do not slide as do most body joints.

Each vertebra has two other sets of joints, called facet joints. As best shown in FIG. 2A, the facet joints are located at the back of the spine (posterior). There is one facet joint on each lateral side (right and left). One pair of facet joints faces upward (called the superior articular facet) and the other pair of facet joints faces downward (called the inferior articular facet). The inferior and superior facet joints mate, allowing motion (articulation), and link vertebrae together. Facet joints are positioned at each level to provide the needed limits to motion, especially to rotation and to prevent forward slipping (spondylolisthesis) of that vertebra over the one below.

In this way, the spine accommodates the rhythmic motions required by humans to walk, run, swim, and perform other regular movements. The intervertebral discs and facet joints stabilize the segments of the spine while preserving the flexibility needed to turn, look around, and get around.

The vertebrae are generally categorized by their location on the spine, as generally shown in FIG. 1. The cervical vertebrae are generally in the head/neck region, and are designated C1 to C7. The thoracic vertebrae are generally in chest/upper back region, and are designated T1 to T12. The lumbar vertebrae are generally in lower back region, and are designated L1 to L5. The sacral vertebrae are generally in the pelvic region, and are designated S1 to S5. There are also the coccygeal vertebrae, so the so-called “tail bone.”

As FIG. 2A shows, all peripheral nerves can be traced back (distally toward the spinal column) to one or more of the spinal nerve roots in either the cervical, thoracic, lumbar, or sacral regions of the spine. The spinal nerves begin as roots at the spine, and form trunks that divide by divisions or cords into branches that innervate skin and muscles.

Facet joints are in almost constant motion with the spine. Facet joints quite commonly simply wear out or become degenerated in many patients. When facet joints become worn or torn, the cartilage may become thin or disappear, and there may be a reaction of the bone of the joint underneath producing overgrowth of bone spurs and an enlargement of the joints. Degenerative changes in the disc can also occur, in turn leading to further arthritic changes in the facet joint and vice versa. Regions of mechanical pain develop (see FIG. 3) where the facet joints rub against each other, as well as along the discs. The joints are then said to have arthritic (literally, joint inflammation-degeneration) changes, or osteoarthritis, that can produce considerable back pain on motion. This condition may also be referred to as “facet joint disease” or “facet joint syndrome”. Further, a protective reflex arrangement arises when the facets are inflamed, which causes the nearby muscles that parallel the spine to go into spasm. Inflamed facet joints therefore can cause crooking and out-of posture of the back, along with powerful muscle spasm.

Degenerative changes in the spine can adversely affect the ability of each spinal segment to bear weight, accommodate movement, and provide support. When one segment deteriorates to the point of instability, it can lead to localized pain and difficulties (as FIG. 3 shows). Segmental instability allows too much movement between two vertebrae. The excess movement of the vertebrae can cause pinching or irritation of nerve roots. It can also cause too much pressure on the facet joints, leading to inflammation. It can cause muscle spasms as the paraspinal muscles try to stop the spinal segment from moving too much. The instability eventually results in faster degeneration in this area of the spine.

Degenerative changes in the spine can also lead to kyphosis (see FIG. 4A) and scoliosis (see FIG. 4B), where the spine has an abnormal curve. Degenerative changes in the spine can also lead to spondylolysis and spondylolisthesis. As FIG. 5 shows, spondylolisthesis is the term used to describe when one vertebra slips forward on the one below it. This usually occurs because there is a spondylolysis (defect) in the vertebra on top. When a spondylolisthesis occurs, the facet joint can no longer hold the vertebra back. The intervertebral disc may slowly stretch under the increased stress and allow the upper vertebra to slide forward.

An untreated persistent, episodic, severely disabling back pain problem can easily ruin the active life of a patient. The total health care expenditures for treating back pain the United States in 1998 were about $26.3 billion. This was three times higher than the total cost of treating all cancer.

In many instances, pain medication, splints, or other normally-indicated treatments can be used to relieve intractable pain in a joint. However, in for severe and persistent problems that cannot be managed by these treatment options, degenerative changes in the spine may require a bone fusion surgery to stop both the associated disc and facet joint problems.

A fusion is an operation where two bones, usually separated by a joint, are allowed to grow together into one bone. The medical term for this type of fusion procedure is arthrodesis.

Lumbar fusion procedures have been increasingly used in the treatment of pain and the effects of degenerative changes in the lower back. A lumbar fusion is a fusion in the S1-L5-L4 region in the spine. The number of lumbar fusions performed in the United States has more than tripled since the early 1990's. Medicare now spends more than $600 million a year on lumbar fusion procedures.

During a spinal fusion, a bone graft is used to join two or more vertebrae. The vertebrae grow together during the healing process, creating a solid piece of bone. The bone graft helps the vertebrae heal together, or fuse. The bone graft is usually taken from the pelvis at the time of surgery. However, some surgeons prefer to use bone graft from a bone bank (called allograft).

Conventionally, the surgeon can use an open anterior (from the front) surgical approach, an open posterior (from the back) surgical approach, or a combined approach to lumbar fusion surgery.

The anterior interbody approach allows the surgeon to remove the intervertebral disc from the front and place the bone graft between the vertebrae. This operation is usually done by making an incision in the abdomen, just above the pelvic bone. The organs in the abdomen, such as the intestines, kidneys, and blood vessels, are moved to the side to allow the surgeon to see the front of the spine. The surgeon then locates the problem intervertebral disc and removes it. Bone graft is placed into the area between the vertebrae where the disc has been removed.

The posterior approach is done from the back of the patient. This approach can be just a fusion of the vertebral bones or it can include removal of the problem disc. If the disc is removed, it is replaced with a bone graft. With a posterior approach, an incision is made in the middle of the lower back over the area of the spine that is going to be fused. The muscles are moved to the side so that the surgeon can see the back surface of the vertebrae. Once the spine is visible, the lamina of the vertebra is removed to take pressure off the dura and nerve roots. This allows the surgeon to see areas of pressure on the nerve roots caused by bone spurs, a bulging disc, or thickening of the ligaments. The surgeon can remove or trim these structures to relieve the pressure on the nerves. Once the surgeon is satisfied that all pressure has been removed from the nerves, a fusion is performed. When operating from the backside of the spine, the most common method of performing a spinal fusion is to place strips of bone graft over the back surface of the vertebrae.

Working between the vertebrae from the back of the patient has limitations. The surgeon is limited by the fact that the spinal nerves are constantly in the way. These nerves can only be moved a slight amount to either side. This limits the ability to see the area. There is also limited room to use instruments and place implants. For these reasons, many surgeons prefer to make a separate incision in the abdomen and actually perform two operations-one from the front of the spine and one from the back. The two operations are usually performed at the same time, but they may be done several days apart.

In the past, spinal fusions of the lumbar spine were performed without any internal fixation. The surgeon simply roughed up the bone, placed bone graft material around the vertebrae, and hoped the bones would fuse. Sometimes, patients were placed in a body cast to try to hold the vertebrae still while healing.

Instrumented spine fusion procedures have been developed. Typical instrumented procedures employ, e.g., specially designed pedicle screws, plates, and rods to hold the vertebrae in place while the spine fusion heals (see FIGS. 6A and 6B). A combination of metal screws and rods (hardware) creates a solid “brace” that holds the vertebrae in place. Special screws called “pedicle screws” are placed through the pedicle bone on the back of the spinal column. The screw inserts through the pedicle and into the vertebral body, one on each side. The screws grab into the bone of the vertebral body, giving them a good solid hold on the vertebra. Once the screws are placed they are attached to metal rods that connect all the screws together. When everything is bolted together and tightened, this creates a stiff metal frame that holds the vertebrae still so that healing can occur. The bone graft is then placed around the back of the vertebrae. These devices are intended to stop movement from occurring between the vertebrae. These metal devices give more stability to the fusion site and allow the patient to be out of bed sooner.

Typical instrumented procedures can also employ, e.g., intervertebral fusion cages to perform a spinal fusion between two or more vertebrae (see FIGS. 7A and 7B). The intervertebral fusion cage is a hollow cylinder. The cages are made from various materials including metal or carbon graphite fiber. Doctors place bone graft inside the cylinder. The holes in the cage keep the graft in contact with the bony surface of the vertebrae. This ensures that the bone graft unites with the vertebrae, forming a solid fusion.

The cage helps in several ways. The solid cage separates and holds two vertebrae apart. This makes the opening around the nerve roots bigger, relieving pressure on the nerves. As the vertebrae separate, the ligaments tighten up, reducing instability and mechanical pain. The cage also replaces the problem disc while holding the two vertebrae in position until fusion occurs.

Instrumented spine fusion stands out as a uniquely costly enterprise. Multi-level rigid instrumented stabilizations may cost as much as $80,000 and as much as half of the surgical cost can be attributed to instrumentation alone. Further, invasive open surgical techniques (anterior and/or posterior) are required to install the instrumentation.

Like all invasive open surgical procedures, operations on the spine risk infections and require hospitalization. Most patients are able to return home when their medical condition is stabilized, which is usually within one week after fusion surgery. Surgery of the spine continues to be a challenging and difficult area. The vertebrae are small, so there is not much room to place small instruments. Also, many nerves can get in the way of putting screws into the vertebral body. And a large amount of stress is put on the lower back, so finding a metal device that is able to hold the bones together can be difficult.

### SUMMARY OF THE INVENTION

The invention provides systems, devices, and surgical procedures to treat degenerative changes in the spine by performing arthrodesis between vertebrae in the spine using minimally invasive instrumentation and techniques.

One aspect of the invention provides a systems and devices for achieving percutaneous fusion of the spine. The systems and devices comprise a first instrumentation component that is sized and configured to achieve posterior percutaneous transpedicular access to an interior of a first targeted vertebral body through a pedicle of the vertebra. The systems and devices comprise a second instrumentation component that is sized and configured to achieve percutaneous cephalad trans-disc access to an interior of a second targeted vertebral body at a next adjacent superior level to the first targeted vertebral body. The systems and devices comprise a third instrumentation component that is sized and configured to achieve percutaneous disc cavity creation comprising a device for forming an enlarged cavity in the intervertebral disc space between the first and second targeted vertebral bodies. The systems and devices comprise a fourth instrumentation component that is sized and configured to achieve percutaneous disc cavity support comprising a support matrix placed in the enlarged cavity formed by the third instrumentation component and that is sized and configured to separate and hold the first and second vertebral bodies apart, to thereby distract nerve roots and relieve pressure on the nerves, and a device for conveying in a percutaneous manner a volume of a filling material into the support matrix that, over time, hardens to promote fusion of the targeted first and second vertebral bodies.

Another aspect of the invention provides methods for achieving percutaneous fusion of the spine. The methods comprise:

(i) percutaneously manipulating instrumentation to achieve posterior percutaneous transpedicular access to an interior of a first targeted vertebral body through a pedicle of the vertebra,

(ii) percutaneously manipulating instrumentation through the percutaneous transpedicular access achieved during (i), to achieve percutaneous cephalad trans-disc access to an interior of a second targeted vertebral body at a next adjacent superior level to the first targeted vertebral body,

(iii) percutaneously manipulating instrumentation through the percutaneous transpedicular access achieved during (i) and the percutaneous cephalad trans-disc access achieved during (ii), to achieve percutaneous disc cavity creation comprising forming an enlarged cavity in the intervertebral disc space between the first and second targeted vertebral bodies, and

(iv) percutaneously manipulating instrumentation through the percutaneous transpedicular access achieved during (i) and the percutaneous cephalad trans-disc access achieved during (ii), to achieve percutaneous disc cavity support comprising placing a support matrix in the enlarged cavity formed during (iii) that is sized and configured to separate and hold the first and second vertebral bodies apart, to thereby distract nerve roots and relieve pressure on the nerves, and conveying a volume of a filling material into the support matrix that, over time, hardens to promote fusion of the targeted first and second vertebral bodies.

Other objects, advantages, and embodiments of the invention are set forth in part in the description which follows, and in part, will be obvious from this description, or may be learned from the practice of the invention.

### BRIEF DESCRIPTION OF THE DRAWINGS

FIG. 1 is an anatomic view of a human spine.

FIG. 2A is an anatomic view of the lower back region of the spine, showing the lumbar vertebrae L2 to L5, the sacral vertebrae S1 to S5, and the coccygeal vertebrae.

FIG. 2B is an anatomic top view of a vertebral body taken generally along line 2B-2B in FIG. 2A.

FIG. 3 is an anatomic view of the lower back region of the spine as shown in FIG. 2A, showing localized regions of mechanical pain that can develop as a result of facet joint and disc degeneration.

FIGS. 4A and 4B are diagrammatic anatomic views of spine deformation can occur as a result of facet joint and disc degeneration, respectively, kyphosis and scoliosis.

FIG. 5 is a diagrammatic anatomic view showing spondylolisthesis, which is when one vertebra slips forward on the one below it.

FIGS. 6A and 6B are diagrammatic anatomic views showing typical instrumentation, e.g., specially designed pedicle screws, plates, and rods, to hold the vertebrae in place while conventional spine fusion heals.

FIGS. 7A and 7B are diagrammatic views showing a typical intervertebral fusion cage to perform a conventional spinal fusion between two or more vertebrae.

FIGS. 8A to 8E show the components of a system for achieving minimally invasive lumbar fusion, and particular, for achieving percutaneous lumbar fusion.

FIGS. 9A(1)/(2) to 9E(1)/(2) show representative embodiments of an expandable bone drilling unit that can form a component part of the system shown in FIG. 8A.

FIGS. 10A and 10B show representative embodiments of a self-expandable support matrix that can form a component part of the system shown in FIG. 8A.

FIGS. 11A and 11B show representative embodiments of a bone filler delivery assembly for conveying a bone filling material into the self-expandable support matrix shown in FIGS. 10A and 10B, and which can form a component part of the system shown in FIG. 8A.

FIGS. 12A to 12H show representative embodiments of a cast-in-place support matrix that can form a component part of the system shown in FIG. 8A, including techniques for its manipulation.

FIGS. 13A to 13I show techniques for manipulating a first instrumentation component 12 that forms a part of the system shown in FIG. 8A to achieve posterior percutaneous transpedicular access to a first targeted vertebral body.

FIGS. 14A to 14E show techniques for manipulating a second instrumentation component 14 that forms a part of the system shown in FIG. 8A to achieve percutaneous cephalad trans-disc access to a second targeted vertebral body via the percutaneous transpedicular access to the first targeted vertebral body shown in FIGS. 13A to 13I.

FIGS. 15A to 15E show techniques for manipulating a third instrumentation component 16 that forms a part of the system shown in FIG. 8A to achieve percutaneous disc cavity creation between the first and second targeted vertebral bodies shown in FIGS. 14A to 14E.

FIGS. 16A to 16E show techniques for manipulating a fourth instrumentation component 18 that forms a part of the system shown in FIG. 8A to achieve percutaneous disc cavity support.

FIGS. 17A to 17D show techniques for completing minimally invasive lumbar fusion, and particular, for achieving percutaneous lumbar fusion, as shown in the previous drawings.

FIGS. 18A and 18B show the results of a procedure of treating three adjacent vertebral bodies S1, L5, and L4, each with percutaneous, bilateral transpedicular accesses, using the instrumentation components and techniques shown in the previous drawings, and also showing, following use of the instrumentation components and the techniques described above, the installation of pedicle screws, plates, and rods to hold the targeted vertebrae in place while the spine fusion heals.

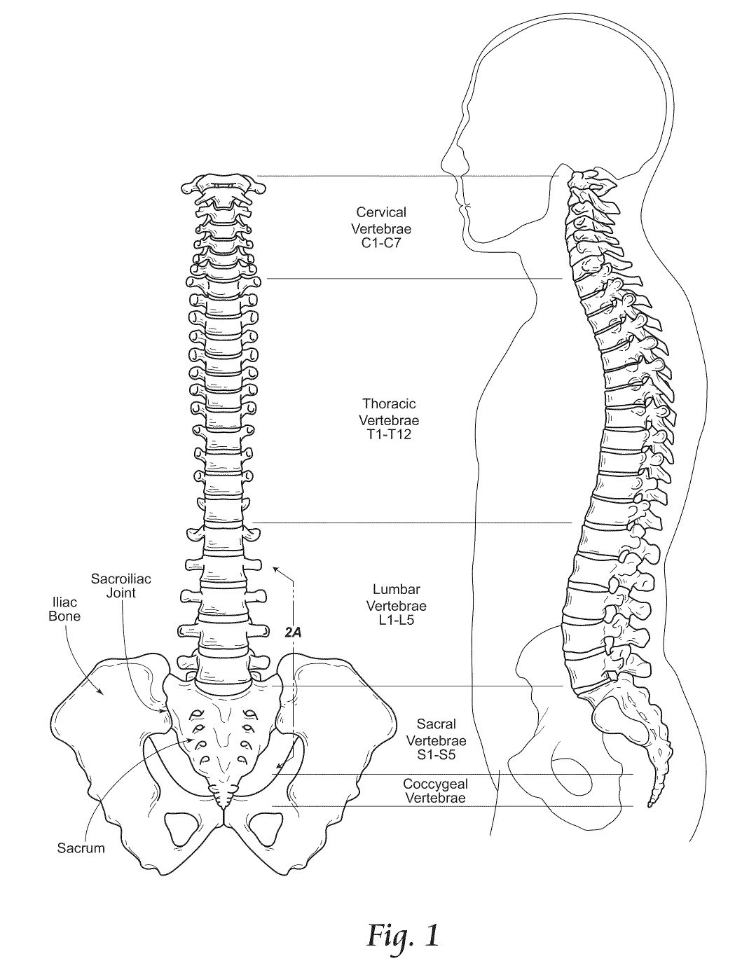

### DESCRIPTION OF THE PREFERRED EMBODIMENTS

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims. While the present invention pertains to systems, devices, and surgical techniques applicable at virtually all spinal levels, the invention is well suited for achieving fusion at the S1-L5-L4 spinal level. It should be appreciated, however, the systems, device, and methods so described are not limited in their application to lumbar fusion and are applicable for use in treating different types of spinal problems.

### I. Anatomy of Lumbar and Sacral Vertebrae

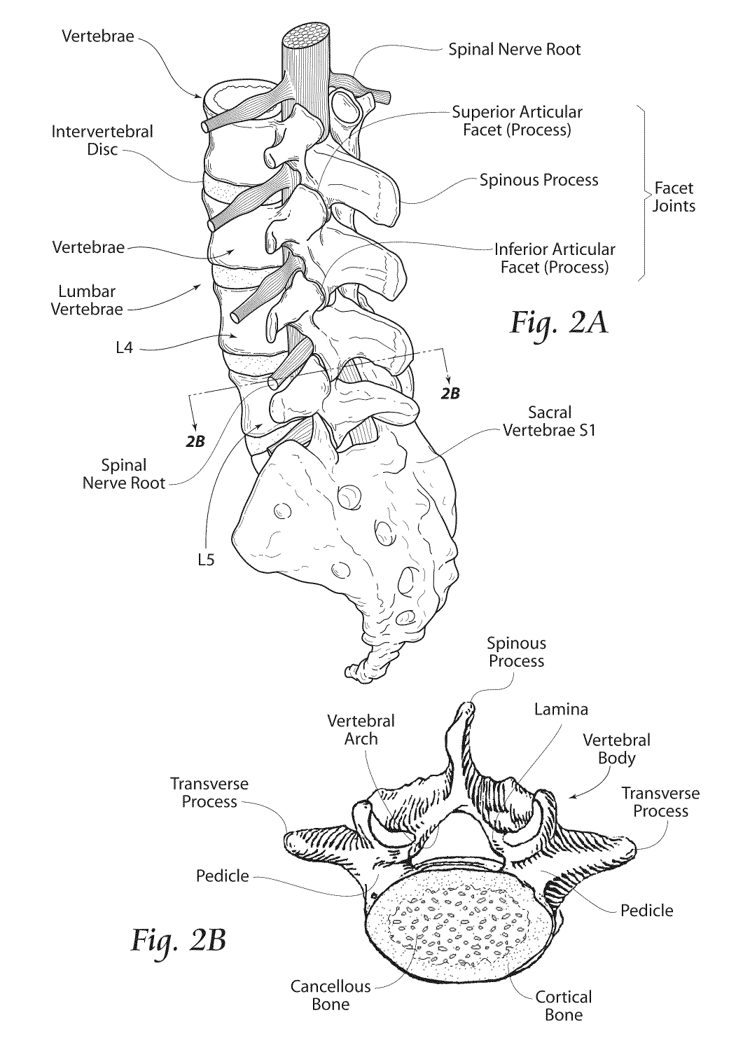

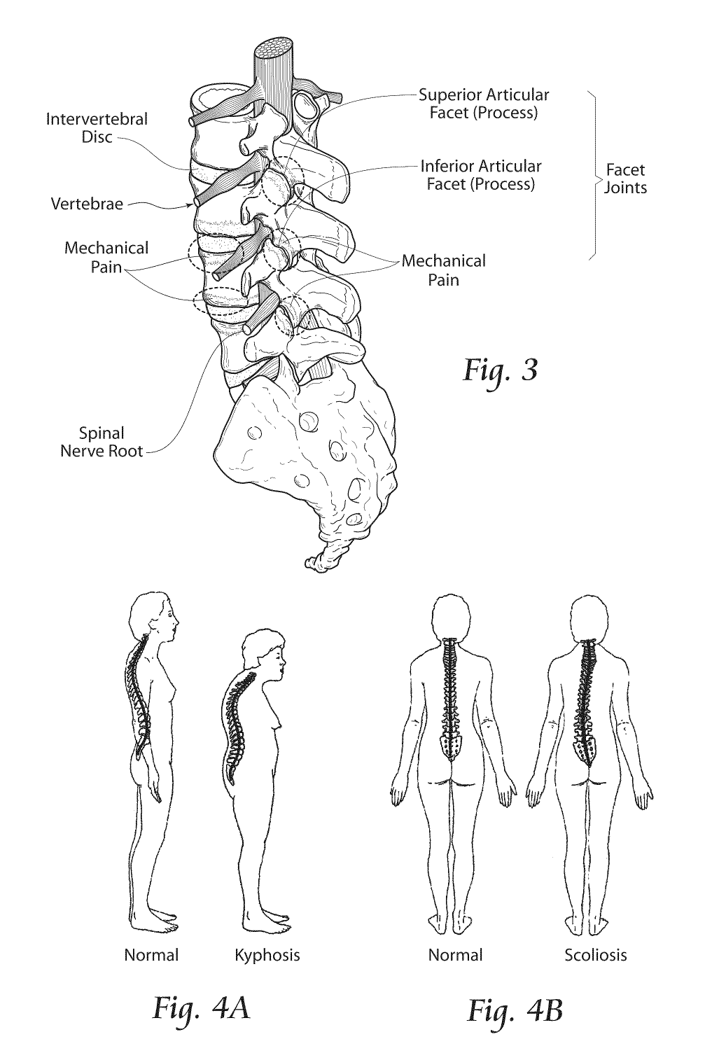

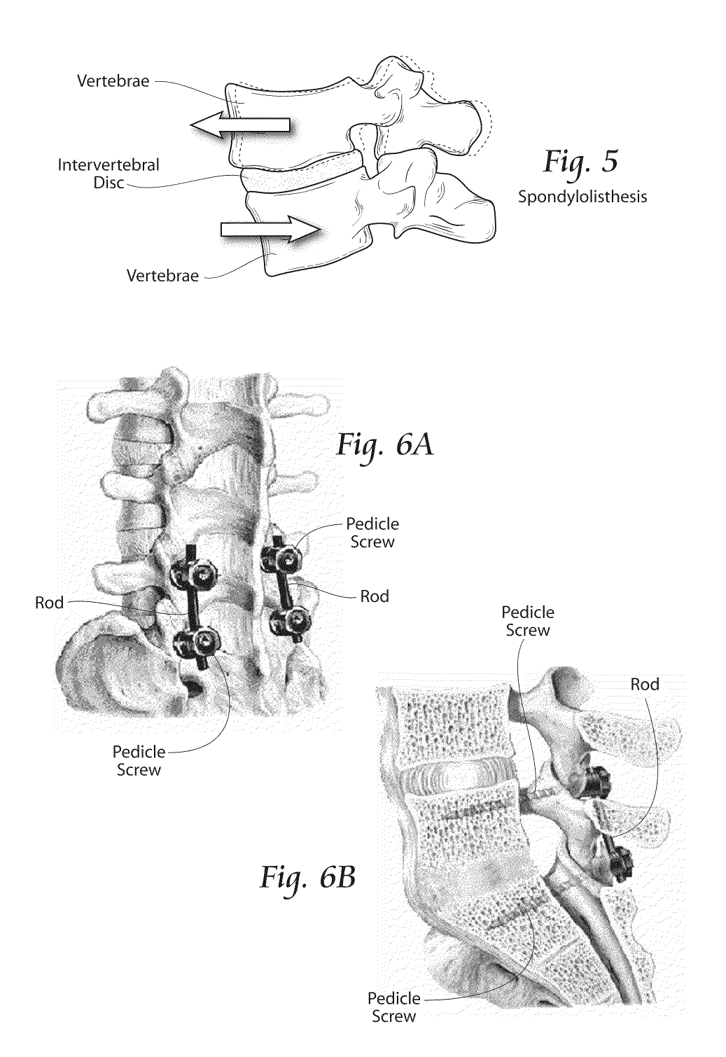

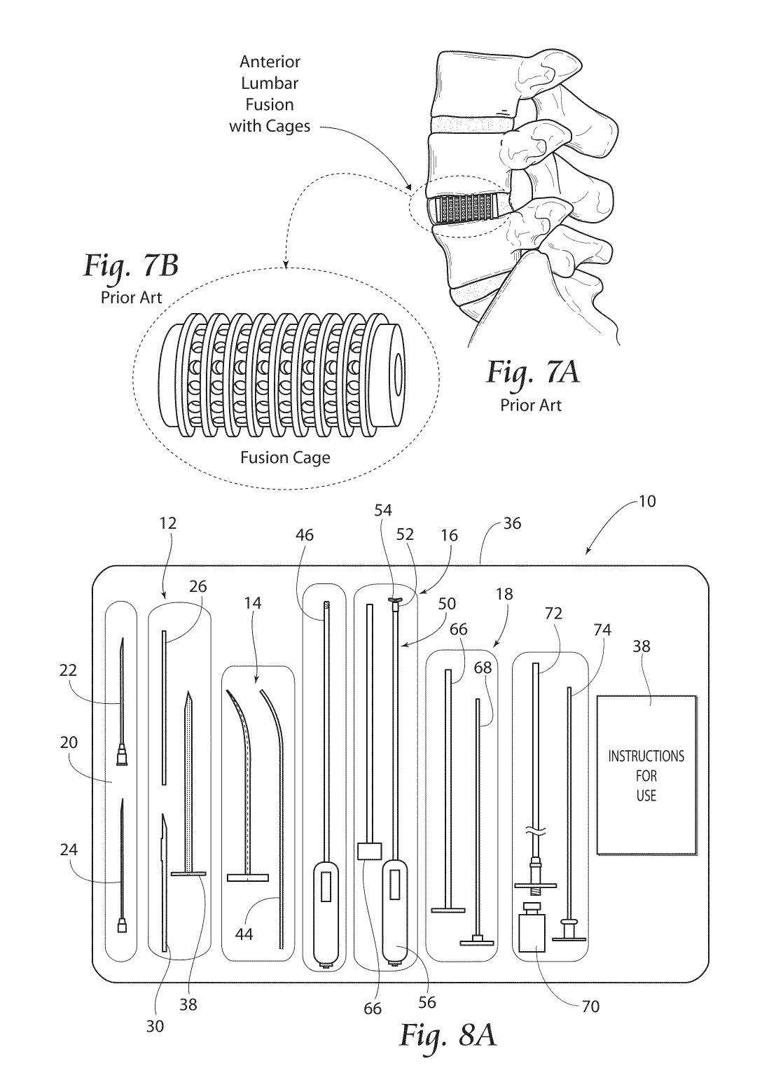

FIG. 2A shows the S1 sacral vertebra and the adjacent fourth and fifth lumbar vertebrae L4 and L5, respectively, in a lateral view (while in anatomic association). The sacral and lumbar vertebrae are in the lower back, also called the “small of the back.”

As is typical with vertebrae, the vertebrae are separated by an intervertebral disc. The configuration of the vertebrae differ somewhat, but each (like vertebrae in general) includes a vertebral body (see FIG. 2B), which is the anterior, massive part of bone that gives strength to the vertebral column and supports body weight. The vertebral arch is posterior to the vertebral body and is formed by the right and left pedicles and lamina. The pedicles are short, stout processes that join the vertebral arch to the vertebral body. The pedicles project posteriorly to meet two broad flat plates of bone, called the lamina.

Seven other processes arise from the vertebral arch. Three processes—the spinous process and two transverse processes—project from the vertebral arch and afford attachments for back muscles, forming levers that help the muscles move the vertebrae. The remaining four processes, called articular processes, project superiorly from the vertebral arch (and are thus called the superior articular processes) and inferiorly from the vertebral arch (and are thus called the inferior articular processes). The superior and inferior articular processes and are in opposition with corresponding opposite processes of vertebrae superior and inferior adjacent to them, forming joints, called facet joints or facets. The facet joints permit gliding movement between the vertebrae. Facet joints are found between adjacent superior and inferior articular processes along the spinal column.

As previously explained, the facet joints can deteriorate or otherwise become injured or diseased, causing lack of support for the spinal column, pain, and/or difficulty in movement.

### II. System for Minimally Invasive Lumbar Fusion

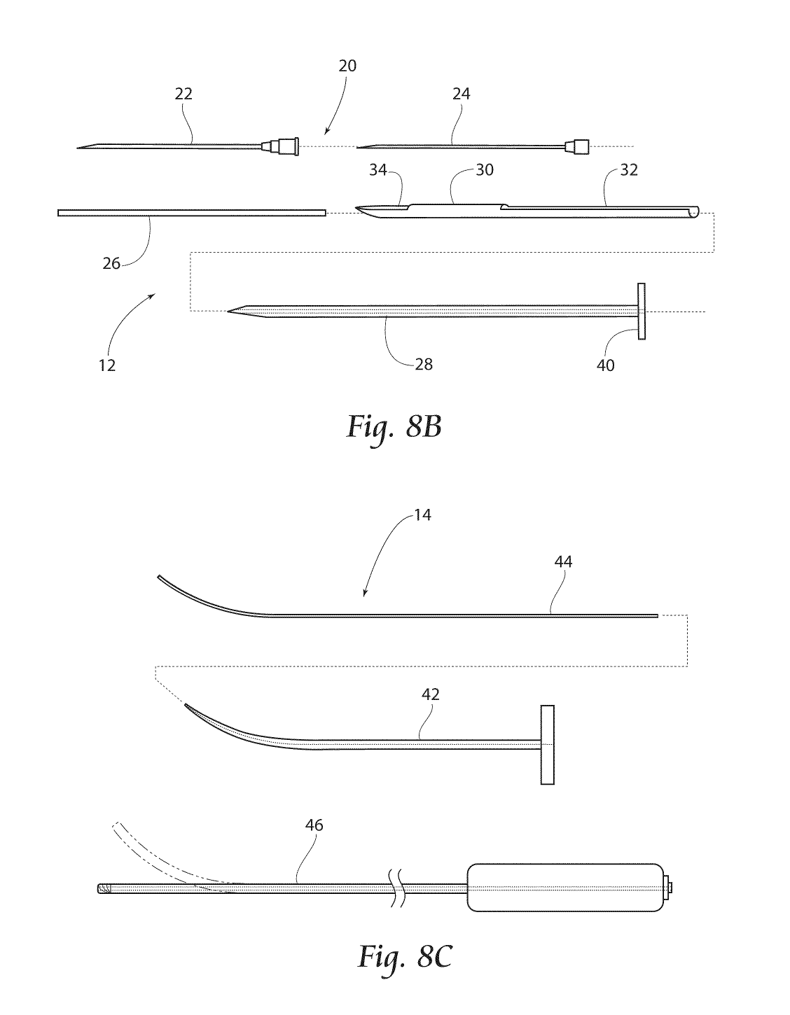

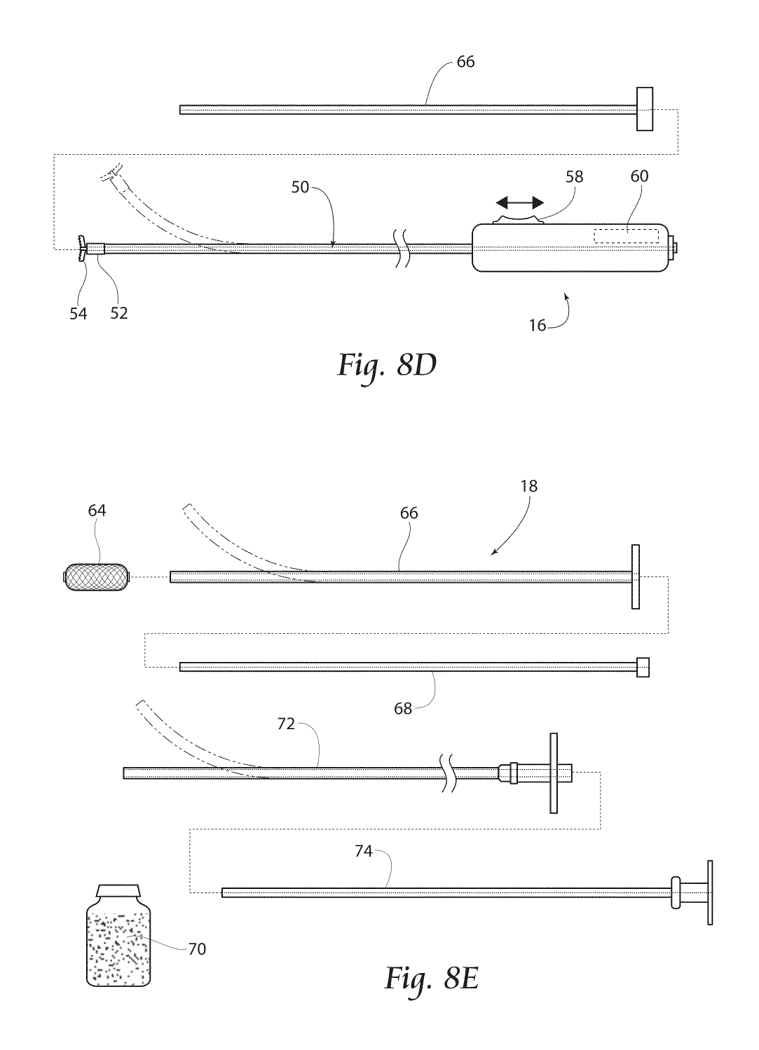

FIG. 8A shows a system 10 for achieving minimally invasive lumbar fusion, and particular, for achieving percutaneous lumbar fusion.

As further shown in FIGS. 8B to 8E, the system 10 includes four instrumentation components 12, 14, 16, and 18.

A. The First Instrument Component

The first instrumentation component 12 (see FIG. 8B) is sized and configured to achieve, in a percutaneous, non-invasive manner, access to the interior of a first targeted vertebral body from the back (posterior) through a pedicle of the vertebra.

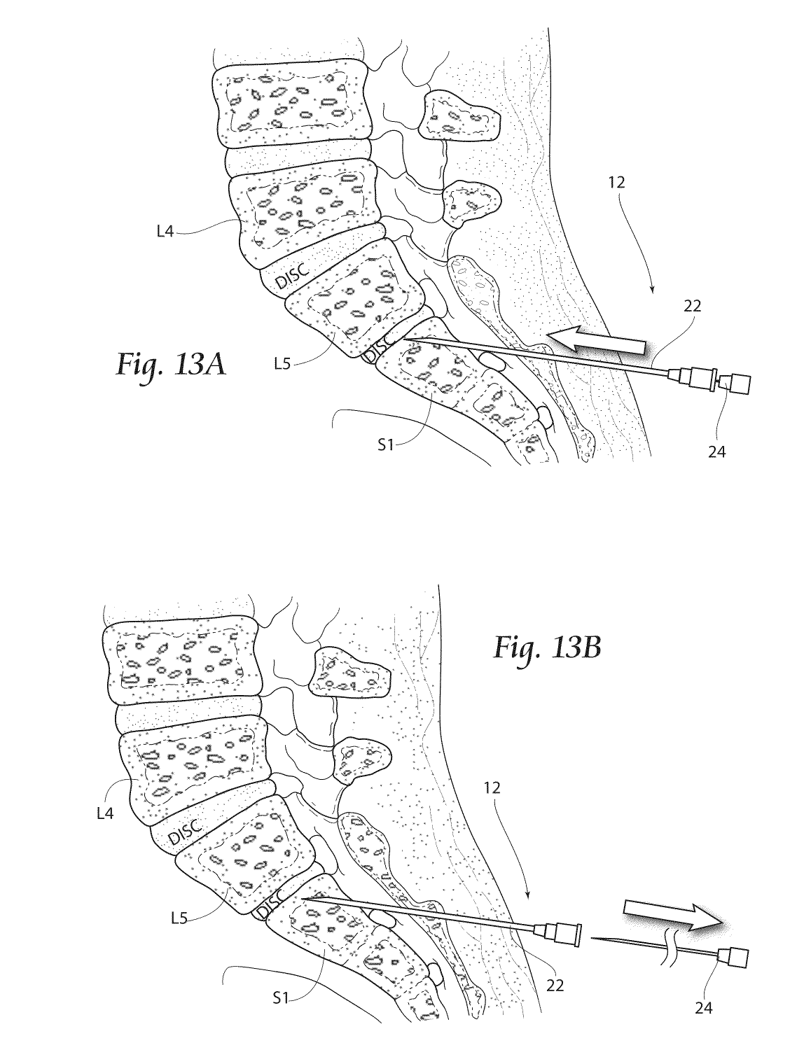

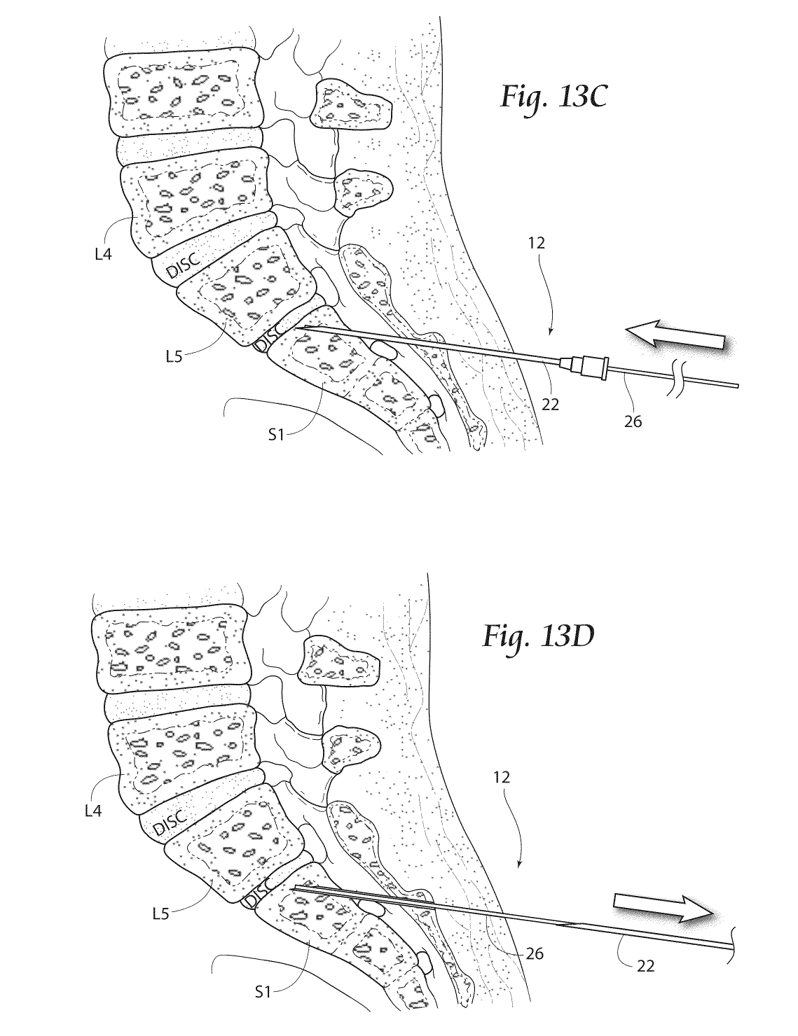

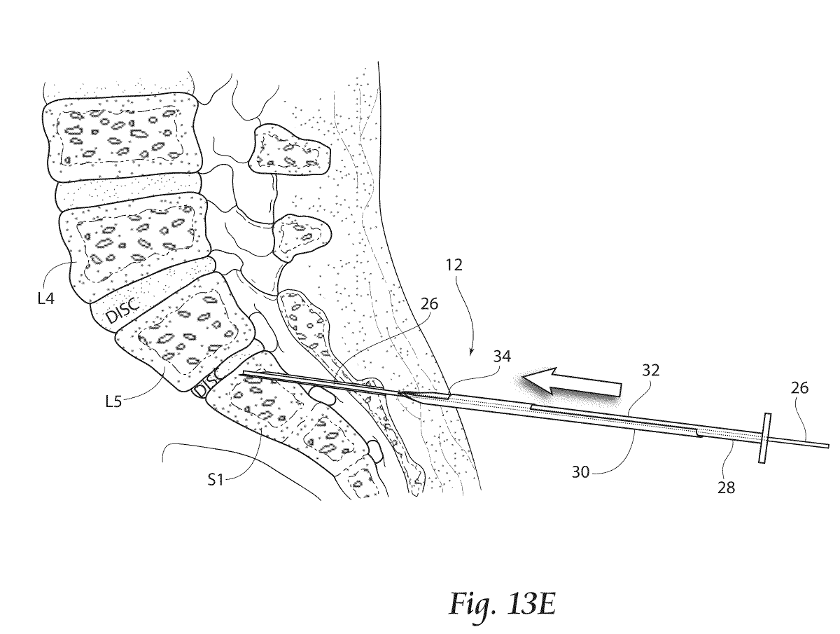

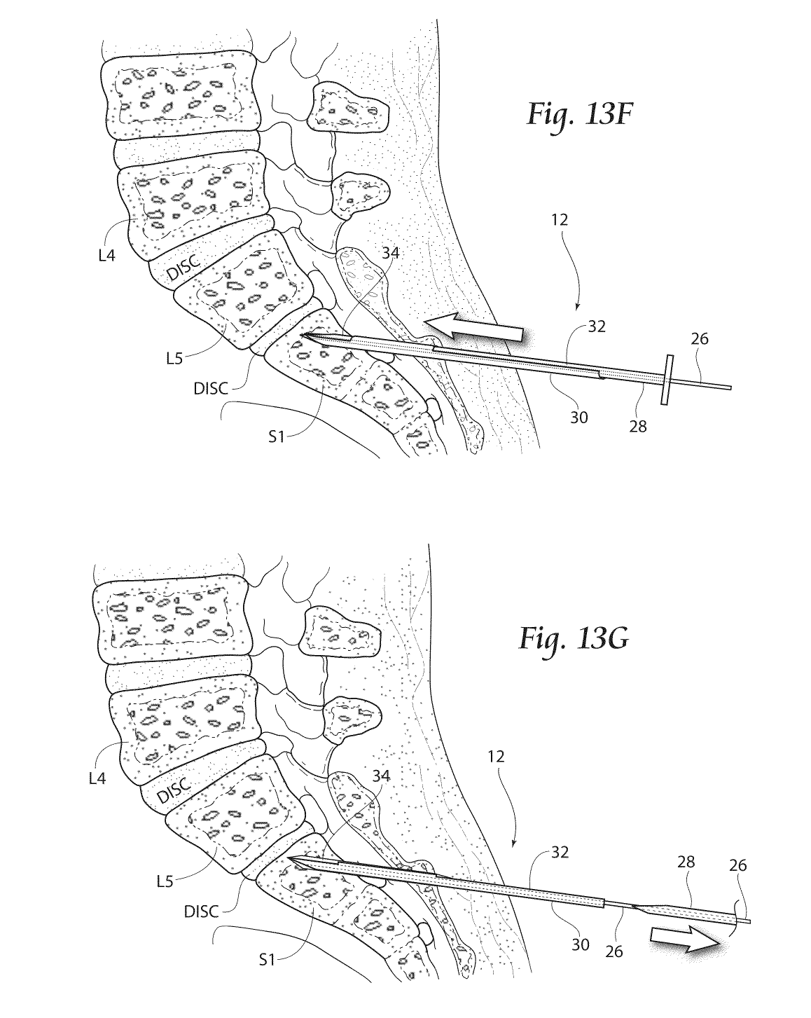

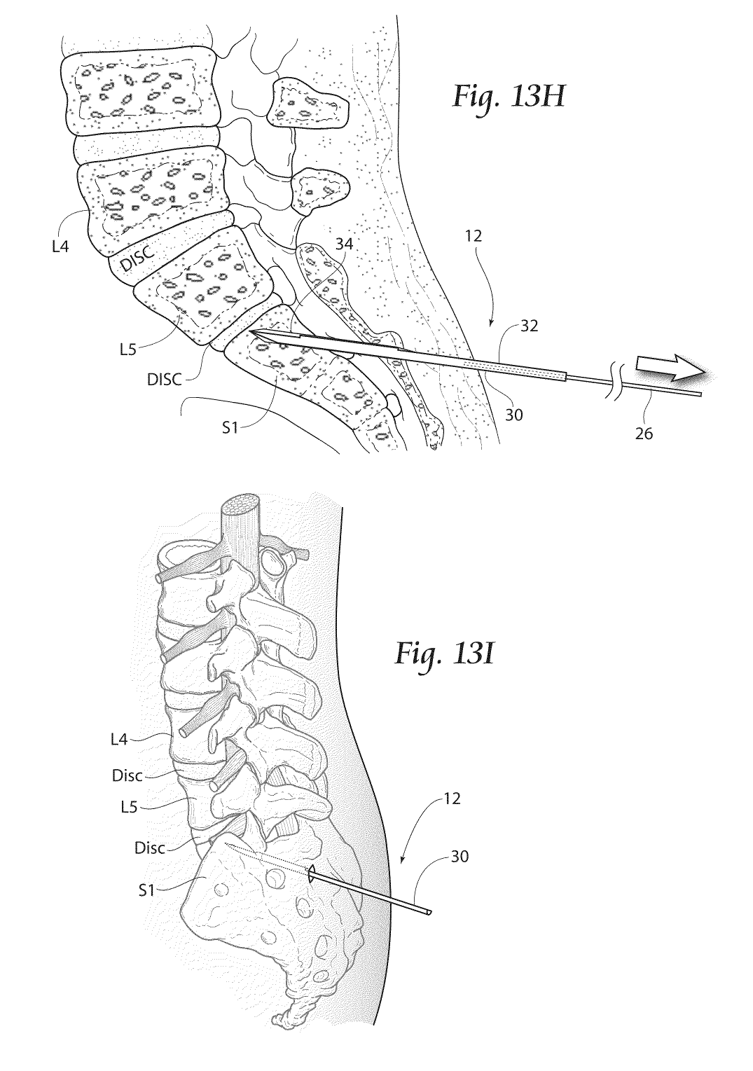

In shorthand, the function of the first instrumentation component 12 will be called posterior percutaneous transpedicular access to the first targeted vertebral body. This is generally shown in FIGS. 13H and 13I, and will be described in greater detail later.

As used herein, “percutaneous” means a medical procedure where access to the vertebra is done via needle-puncture of the skin, rather than by using an “open” approach where inner organs or tissue are exposed (typically with the use of a scalpel).

The first instrumentation component 12 can be variously configured, and representative embodiments are shown in FIG. 8B and will be described in greater detail later.

B. The Second Instrumentation Component

The second instrumentation component 14 (see FIG. 8C) is sized and configured to achieve, in a percutaneous, non-invasive manner—through the posterior percutaneous transpedicular access provided by the first instrumentation component 12—access the interior of a second targeted vertebral body at the next adjacent superior (or cephalad) level. The second instrumentation component 14 is sized and configured to achieve this access to the second targeted vertebral body through the superior end plate of the first targeted vertebral body, then through the intervertebral disc between the first and second targeted vertebral bodies, and then through the inferior (caudal) end plate of the second targeted vertebral body.

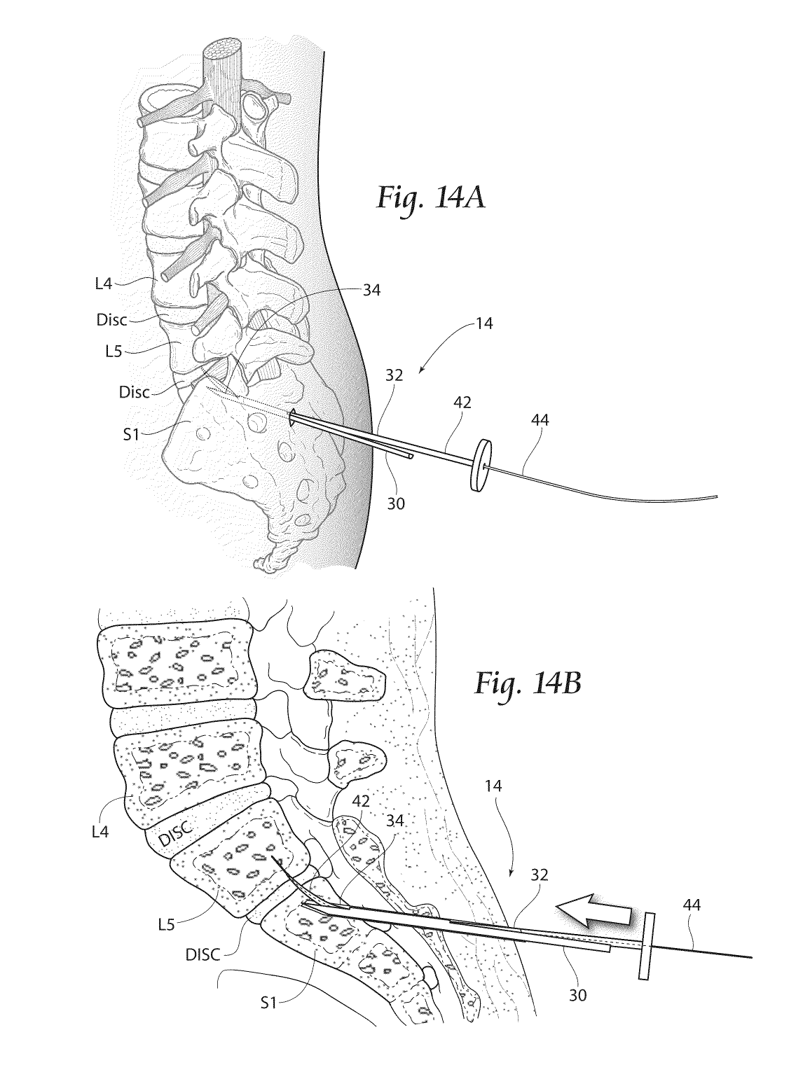

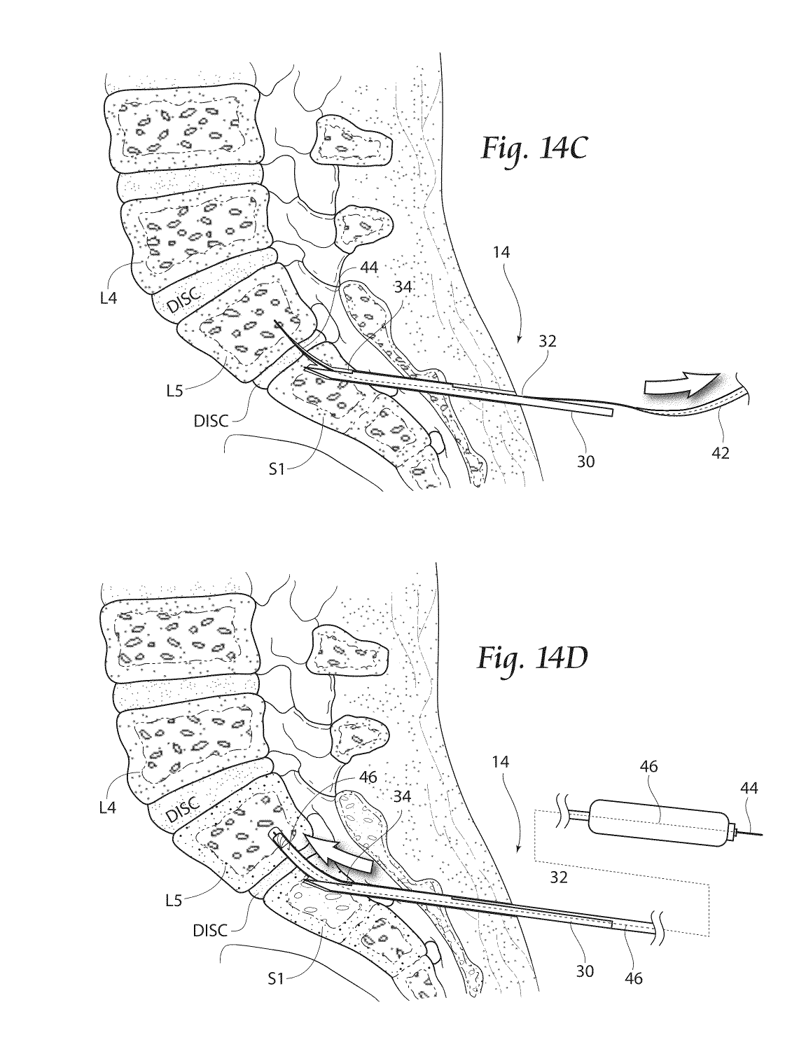

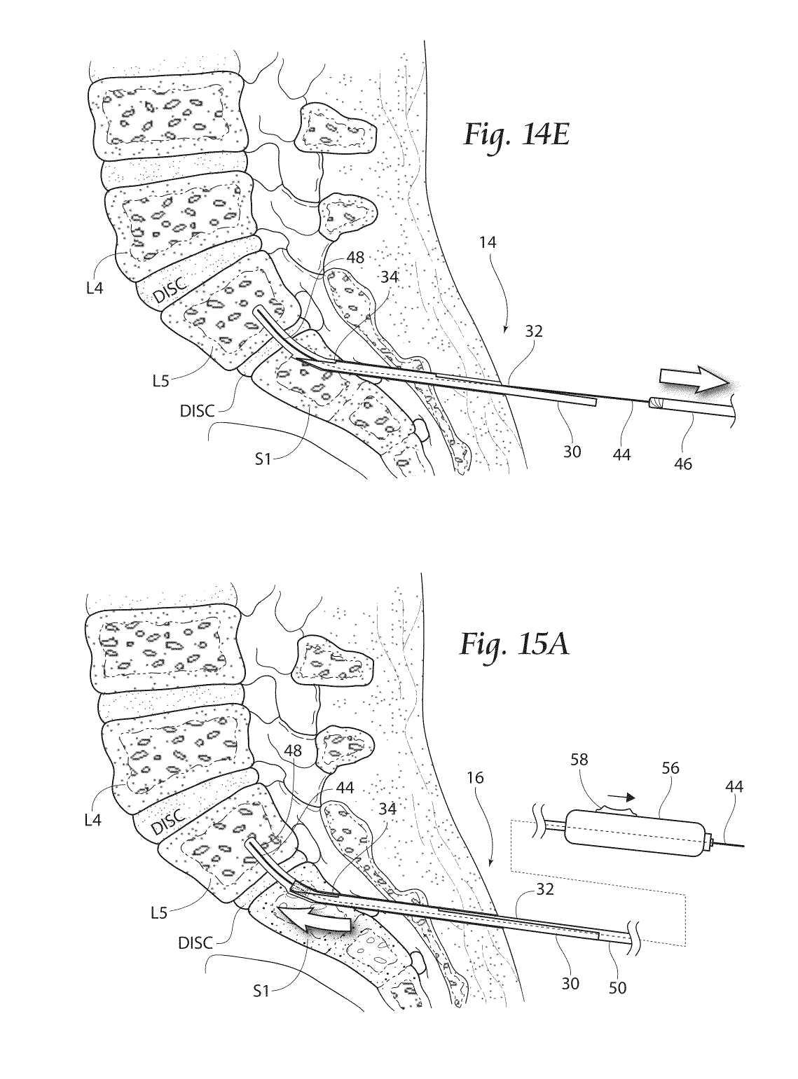

In shorthand, the function of the second instrumentation component 14 will be called percutaneous cephalad trans-disc access to the second targeted vertebral body. This is generally shown in FIG. 14E, and will be described in greater detail later.

The second instrumentation component 14 can be variously configured, and representative embodiments are shown in FIG. 8C and will be described in greater detail later.

C. The Third Instrumentation Component

The third instrumentation component 16 (see FIG. 8D) is sized and configured to form, in a percutaneous, non-invasive manner, an enlarged cavity 62 in the intervertebral disc space between the first and second targeted vertebral bodies. The enlarged cavity 62 desirably also includes regions of removed cortical bone in adjacent regions of the end plates adjoining the intervertebral disc.

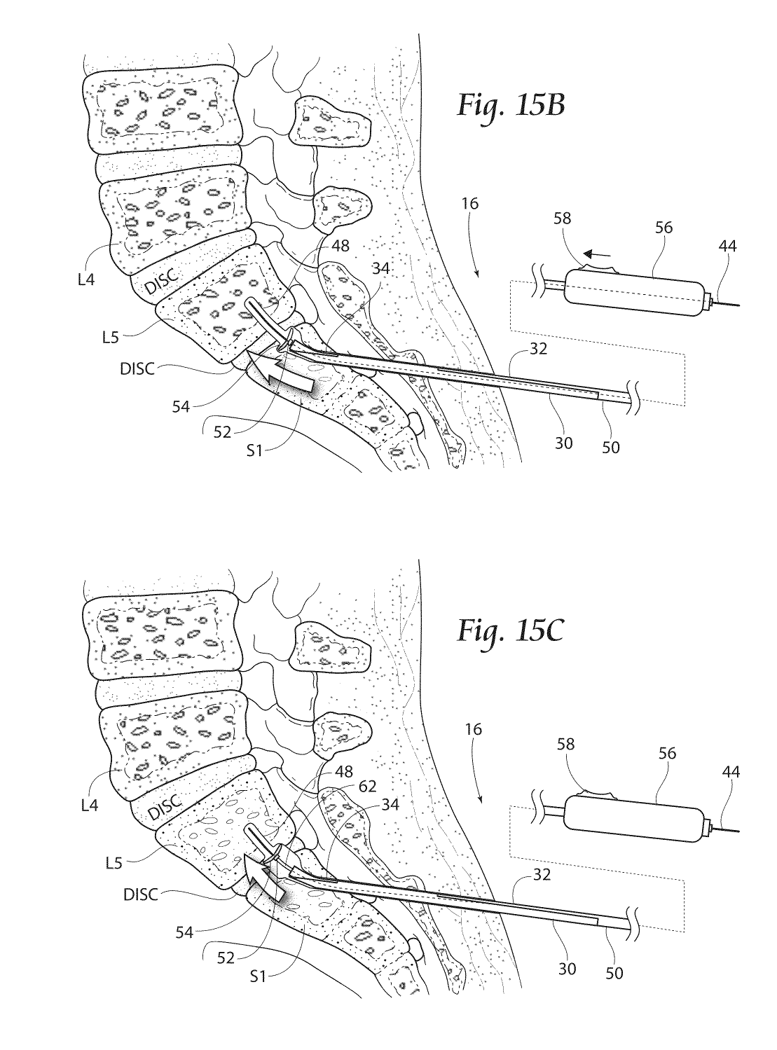

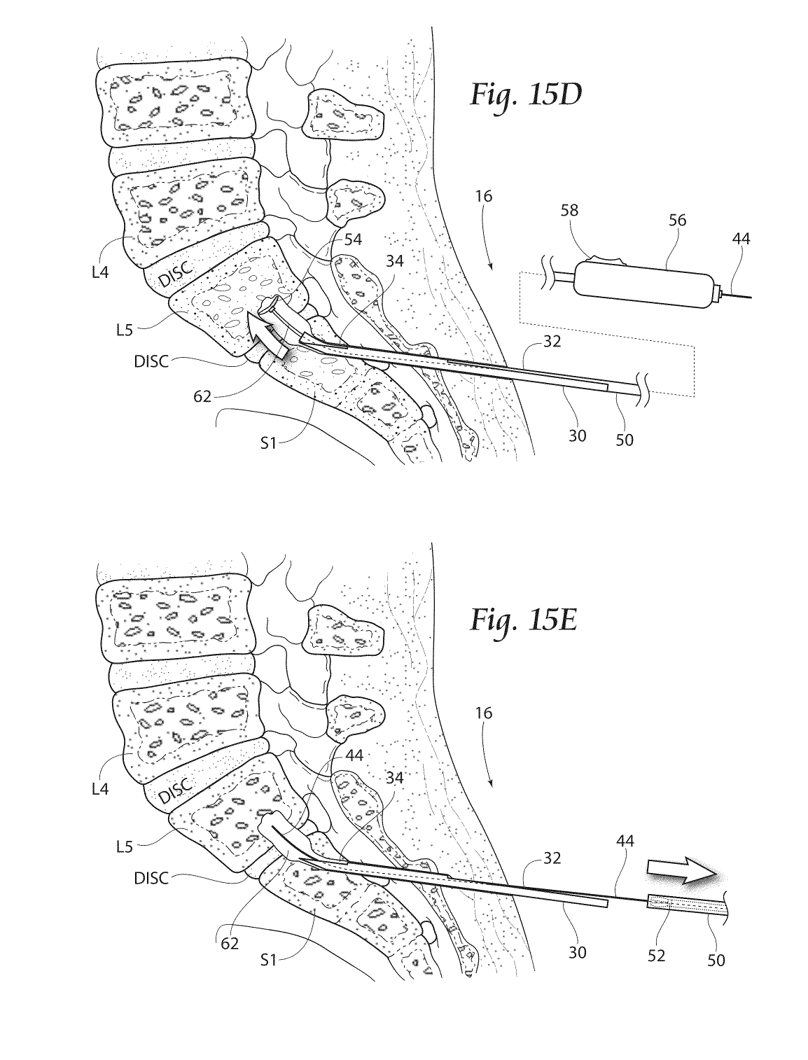

In shorthand, the function of the third instrumentation component 16 will be called percutaneous disc cavity creation. This is generally shown in FIG. 15E, and will be described in greater detail later.

The third instrumentation component 16 can be variously configured, and representative embodiments are shown in FIG. 8D and will be described in greater detail later.

D. The Fourth Instrumentation Component

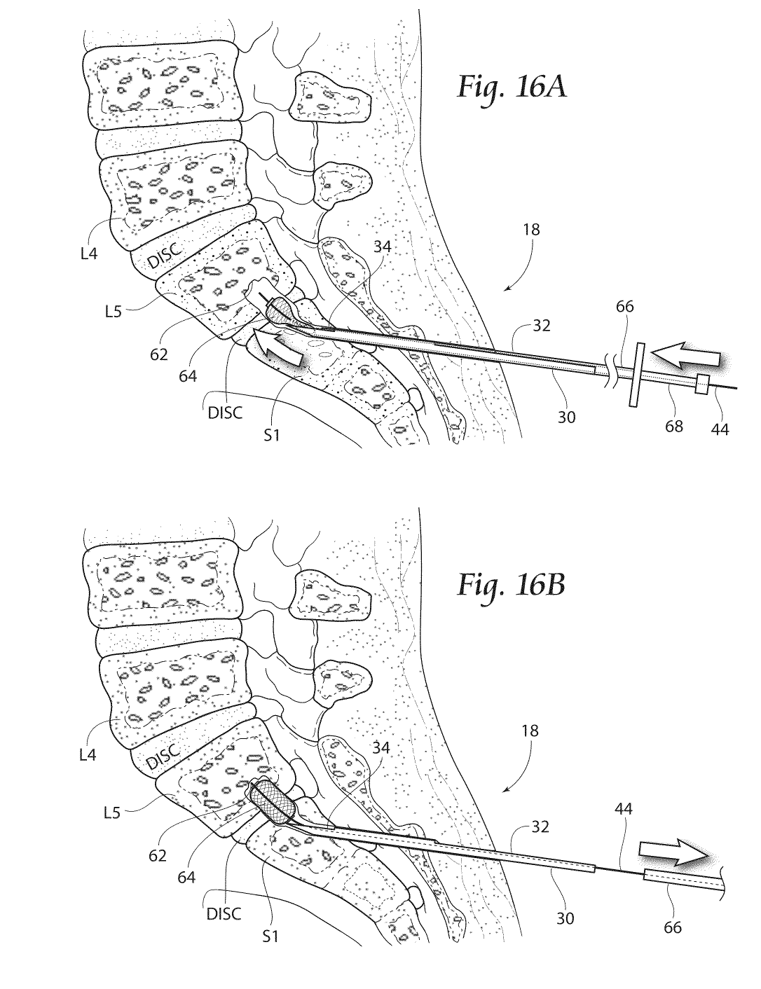

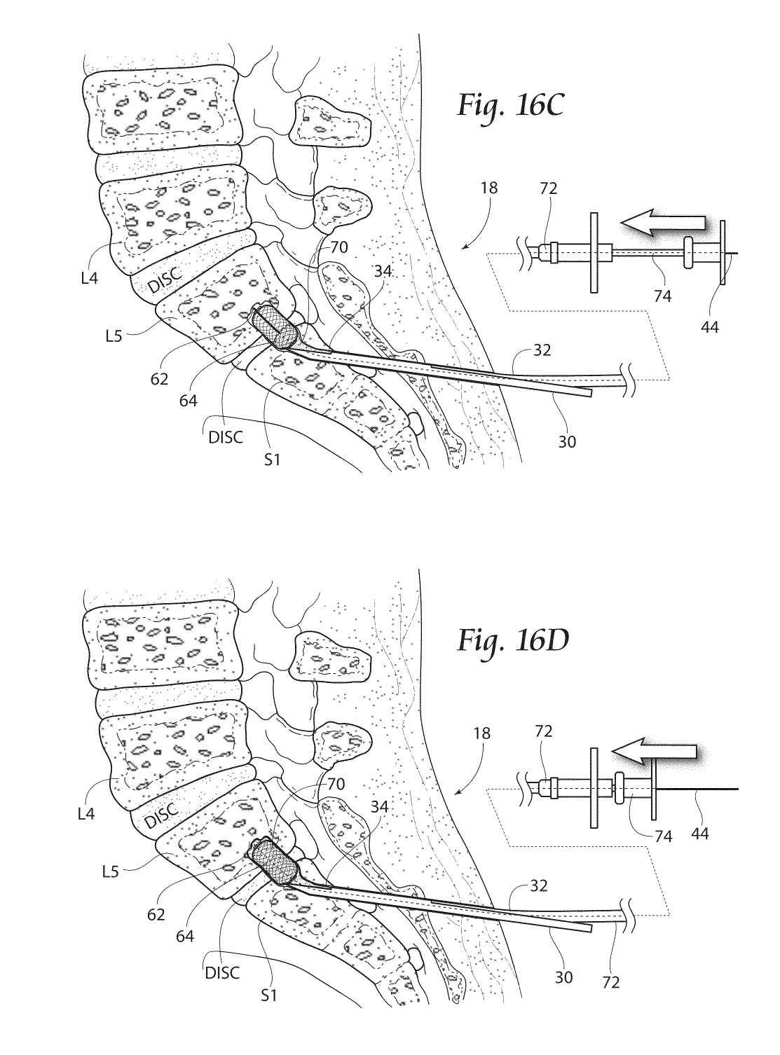

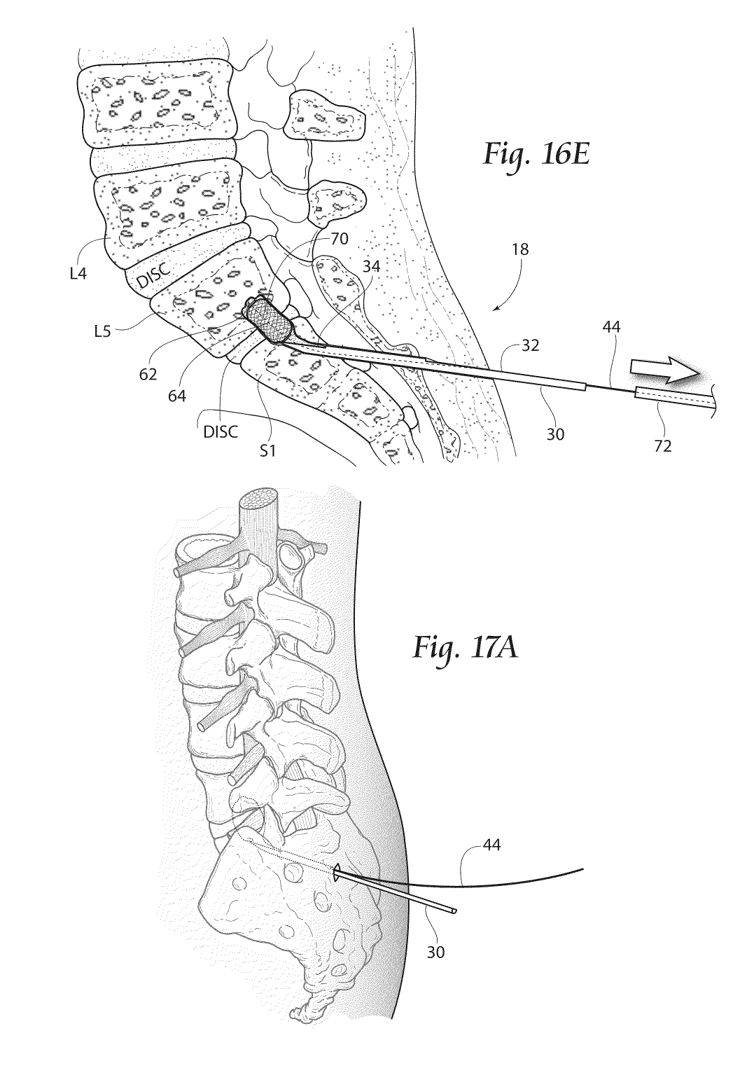

The fourth instrumentation component 18 (see FIG. 8E) is sized and configured to place and position, in a percutaneous, non-invasive manner, a support matrix or structure 64 in the enlarged cavity 62 formed by the third instrumentation component 16. This is generally shown in FIG. 16E, and will be described in greater detail later.

The support matrix or structure 64 is sized and configured to separate and hold the first and second vertebral bodies apart, to thereby distract the nerve roots and relieve pressure on the nerves. The support matrix or structure 64 is also desirably sized and configured to receive, in a percutaneous, non-invasive manner, a volume of a filling material that, over time, hardens to promote fusion of the targeted first and second vertebral bodies. The conveyance of the filling material into the support matrix or structure 64 can also serve to further distract and relieve pain by decompressing nerve roots between the first and second vertebral bodies.

For example, the filling material can comprise a flowable polymer material or a bone graft material that, upon setting, helps the vertebrae heal together, or fuse. In this arrangement, the fourth instrumentation component 18 is sized and configured to convey the filling material into the support matrix or structure 64.

In shorthand, the function of the fourth instrumentation component 18 will be called percutaneous disc cavity support.

The fourth instrumentation component 18 can be variously configured, and representative embodiments are shown in FIG. 8# and will be described in greater detail later.

As shown in FIG. 8A, the various instrumentation components just described can be arranged in one or more prepackage kits 36. The kits 36 also preferably include directions 38 for using the contents of the kits 36 to carry out a desired minimally invasive lumbar fusion, as will now be described in greater detail.

### II. Surgical Techniques for Achieving Minimally Invasive Lumbar Fusion Using the System

A. Achieving Posterior Percutaneous Transpedicular Access to the First Targeted Vertebral Body (e.g., the S1 Vertebra)

In the representative embodiment shown in FIG. 8B, the first instrumentation component 12 includes a spinal needle assembly 20 including a stylus 22 and removable stylet 24; a guide pin or wire 26; a cannulated obturator 28; and a slotted guide tube 30. As shown in FIG. 8B, the guide tube 30 includes a proximal slotted side wall 32 and a distal slotted side wall 34. The slotted guide tube 30 is sized and configured to slip over and be carried by the body of the obturator 28. The slotted guide tube 30 is sized and configured to accommodate percutaneous cephalad trans-disc access to the second targeted vertebral body by the second instrumentation component 14, as will be described in greater detail later.

Representative techniques for manipulating the first instrumentation component 12 are shown in FIGS. 13A to 13H). In this arrangement, the directions 38 for using the first instrumentation component 12 include identifying, e.g., by tactile and radiologic or fluoroscopic techniques, the S1-L5 region in a patient's spine targeted for fusion (as FIG. 2A generally shows). A right pedicle or left pedicle of the S1 vertebra is identified as the targeted access site. Pedicles serve as good fluoroscopic targets.

Under radiologic or fluoroscopic monitoring (see FIG. 13A), the spinal needle assembly 20 (stylet 24 carried within the stylus 22) is advanced through soft tissue down to and into the targeted left or right pedicle. A local anesthetic, for example, lidocaine, may be administered as percutaneous access is achieved by use of the spinal needle assembly 20.

Under radiologic or fluoroscopic monitoring, the spinal needle assembly 20 is further directed through the targeted pedicle to penetrate a distance into the cortical bone in the S1 vertebra (as FIG. 13A shows), desirably without entering cancellous bone.

The stylet 24 of the spinal needle assembly 20 is withdrawn from the stylus 22 (see FIG. 13B). The guide pin 26 is introduced by sliding through the stylus 22 into the cortical bone of the S1 vertebra (see FIG. 13C). The stylus 22 is withdrawn, leaving the guide pin 26 deployed within the cortical bone of the S1 vertebra (see FIG. 13D).

The slotted guide tube 30 is slipped over the body of the obturator 28. A small incision is made in the patient's back around the guide pin 26. The cannulated obturator 28 (carrying the slotted guide tube 30) is passed over the guide pin 26 (see FIG. 13E). Under radiologic or fluoroscopic monitoring, the obturator 28 is twisted while appropriate longitudinal pushing force is applied. Preferably, the obturator 28 includes a handle 40 to facilitate its manipulation.

In response, the obturator 28 rotates and penetrates soft tissue through the incision under radiologic or fluoroscopic monitoring. The handle 40 may be gently tapped, or appropriate additional longitudinal force may be otherwise apply to the obturator 28, to aid advancement of the obturator 28 (and the slotted guide tube 30 it carries) along the guide pin 26 down to the cortical bone entry site on the pedicle.

During advancement, the proximal and distal slotted end walls 32 and 34 of the guide tube 30 are oriented to face in a cephalad direction, i.e. in the direction of the L5 vertebra, as FIG. 13E shows.

Under radiologic or fluoroscopic monitoring, the handle 40 may be further gently tapped, or appropriate additional longitudinal force may be otherwise apply to the obturator 28, to advance the obturator 28 (and the slotted distal side wall 34 of the slotted guide tube 30 it carries) through the pedicle and into the cortical bone of the S1 vertebral body (see FIG. 13F). The obturator 28 has an outside diameter that accommodates transpedicular access without damage or breakage of the pedicle. The orientation of the slotted distal side wall 34 of the slotted guide tube 30 is maintained to face in a cephalad direction toward the L5 vertebra.

The obturator 28 and/or slotted guide tube 30 can by wires be EMG connected to provide intraoperative stimulation of nerve routes, to aid in insertion and positioning.

Under radiologic or fluoroscopic monitoring, the slotted distal side wall 34 of the slotted guide tube 30 is advanced a desired distance through the pedicle into the cortical bone of the S1 vertebra, as FIG. 13F shows. The obturator 28 is withdrawn over the guide pin 26 (see FIG. 13G). The guide pin 26 is withdrawn from the slotted guide tube 30 (see FIG. 13H). The slotted guide tube 30 remains with the proximal slotted wall 32 of the slotted guide tube 30 exposed beyond the incision, oriented in a cephalad direction, as FIG. 13I shows.

Posterior percutaneous transpedicular access to the first targeted vertebral body (i.e., the S1 vertebra) has been achieved (shown FIG. 13I).

B. Achieving Percutaneous Cephalad Trans-Disc Access from the First Targeted Vertebral Body (e.g., the S1 Vertebra) to the Second Targeted Vertebral Body (e.g., the L5 Vertebra)

In the representative embodiment shown in FIG. 8C, the second instrumentation component 14 includes a curved cannulated stylus or suture instrument 42 having a center lumen. The curved cannulated stylus or suture instrument 42 is sized and configured to be passed through the slotted guide tube 30 in a curvilinear path through the slotted proximal and distal side walls 32 and 34 (see FIGS. 14A and B). The geometry of the curvature is selected so that passage of the stylus or suture instrument 42 in the curvilinear path through the slotted guide tube 30 directs the distal end of the stylus or suture instrument 42 through the superior end plate of the first targeted vertebral body (i.e., the S1 vertebra) into and through the intervertebral disc between the first and second targeted vertebral bodies and into and through the inferior (caudal) end plate of the second targeted vertebral body (i.e., the L5 vertebra) (as FIG. 14B shows).

The curvature of the cannulated stylus or suture instrument 42 can be pre-formed or can be set at the instance of use by the incorporation of semi-rigid material that are bendable or deformable to a desired curvature. The curvature of the cannulated stylus or suture instrument 42 can be ascertained, taking into account the morphology and geometry of the site to be treated. The morphology of the local structures can be generally understood by medical professionals using textbooks of human skeletal anatomy along with their knowledge of the site and its disease or injury. The physician is also desirably able to set the curvature desired based upon prior analysis of the morphology of the targeted bone using, for example, plain film x-ray, fluoroscopic x-ray, or MRI or CT scanning.

In the representative embodiment shown in FIG. 8C, the second instrumentation component 14 further includes a flexible or pre-curved guide wire or pin 44 (made, e.g., of stainless steel) having a sharpened tip. The guide wire or pin 44 is sized and configured to be passed through the center lumen of the curved cannulated stylus or suture instrument 42, in the manner that a spinal needle stylet can be passed through a spinal needle stylus. The guide wire or pin 44 can comprise, e.g., a Kirschner wire or K-wire used to hold bone fragments together (pin fixation) or to provide an anchor for skeletal traction. For this reason, the guide wire or pin 44 will be generally referred to as a K-wire, meaning a Kirschner wire or an equivalent of a Kirschner wire.

In the representative embodiment shown in FIG. 8C, the second instrumentation component 14 further includes a cannulated flexible bone drill 46 sized and configured to be passed over the K-wire 44 through the slotted guide tube 30.

Representative techniques for manipulating the second instrumentation component 14 are shown in FIGS. 14A to 14E). In this arrangement, the directions 38 for using the second instrumentation component 14 includes inserting the K-wire 44 into the center lumen of curved cannulated stylus or suture instrument 42, as FIGS. 14A and 14B show). Under radiologic or fluoroscopic monitoring, the curved cannulated stylus or suture instrument 42 (with K-wire 44) is introduced in the curvilinear path through the slotted guide tube 30 and into cortical bone of the first targeted vertebral body (i.e., S1 vertebra). Like a stylet in a spinal needle assembly 20, the sharpened tip of the K-wire 44 within the curved cannulated stylus or suture instrument 42 will penetrate cortical bone in advance of the curved cannulated stylus or suture instrument 42 in response to an applied longitudinal force. Under radiologic or fluoroscopic monitoring, the distal end of the curved cannulated stylet or suture instrument 42 can be directed, due to its preselected curvature, radially out the slotted distal side wall of the slotted guide tube 30, in a path that extends in a cephalad direction through the cortical bone of the end plate of the S1 vertebra, into and through the adjoining disc, and into and through the end plate a desired distance into cortical bone of the next superior vertebral body (L5).

Subsequent withdrawal of the curved cannulated stylus or suture instrument 42 from the K-wire 44 (as FIG. 14C shows) leaves the K-wire 44 and slotted guide tube 30 behind.

Under radiologic or CT monitoring (see FIG. 14D), the flexible drill 46 is passed over the K-wire 44 in the path defined by the K-wire 44 in a cephalad direction through the cortical bone of the end plate of the S1 vertebra, into and through the adjoining disc, and into and through the end plate a desired distance into cortical bone of the next superior vertebral body (L5). As FIGS. 14D and 14E show, the drill 46 clears an access channel 48 through cortical bone and disc tissue, e.g., a 5 mm channel in diameter. The flexible drill 46 is then withdrawn over the K-wire 44, as FIG. 14E shows). Remnants of cortical bone and/or disc tissue can be aspirated from the access channel 48 by suction, if required.

Percutaneous cephalad trans-disc access from the first targeted vertebral body (i.e., the S1 vertebra) to the second targeted vertebral body (i.e., the L5 vertebra) has been achieved.

C. Achieving Percutaneous Disc Cavity Creation.

In the representative embodiment shown in FIG. 8D, the third instrumentation component 16 includes a flexible tissue drilling unit 50. The flexible drilling unit 50 includes a flexible catheter body having a lumen to accommodate passage over the K-wire 44. The flexible drilling unit 50 also includes, at its distal end, a tissue cutter 52. The tissue cutter 52 includes one or more tissue cutting blades 54.

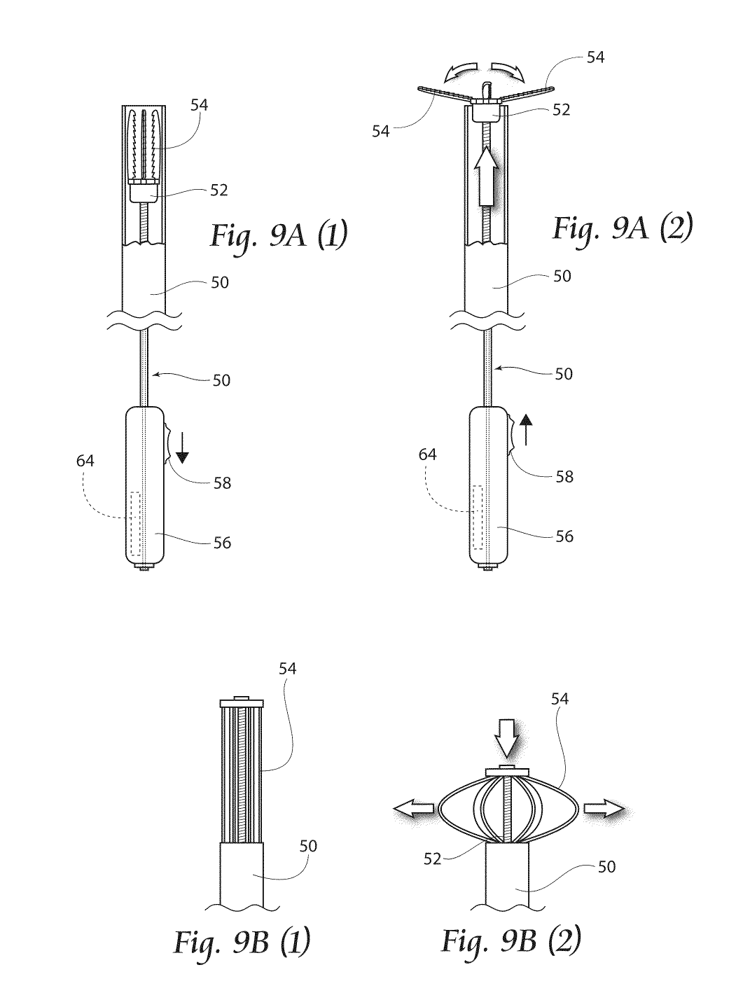

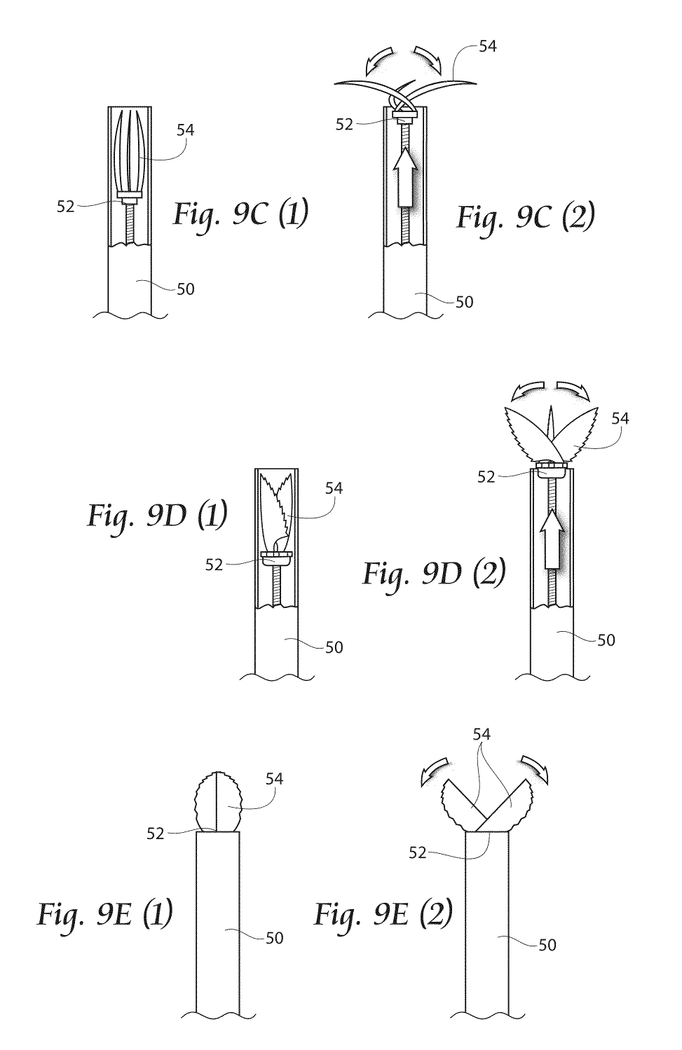

The configuration of the tissue cutting blades 54 can vary, and representative embodiments are shown in FIGS. 9A to 9E. In each representative embodiment (as shown in the 9A(1) to 9E(1) views), the tissue cutting blades 54 are sized and configured to assume a collapsed, lay-flat low profile condition for unobstructed passage with the catheter tube over the K-wire 44 through the confines of the slotted guide tube 30 and formed access channel 48. A handle 56 on the proximal end of the drilling unit 50 aids in the manipulation of the drilling unit 50 over the K-wire 44.

An operator-actuated control 58 on the handle 56 is coupled to the tissue cutter 52. Manipulation of the operator-actuated control 58 (as shown in the 9A(2) to 9E(2) views), e.g. by sliding it forward, causes the tissue cutting blades 54 to expand from their collapsed, lay-flat low profile condition toward a radially extended, deployed condition. When in the radially enlarged condition, the tissue cutting blades 54 assume an increased outer diameter larger than the outer diameter of the catheter tube. Manipulation of the operator-actuated control 58, e.g. by sliding it rearward, causes the tissue cutting blades 54 to return from radially extended, deployed condition toward their collapsed, lay-flat low profile condition (as shown in the 9A(1) to 9E(1) views). The operator is therefore able to enlarge and collapse the cutting blades 54 on demand.

A motor 60 carried by the handle 56 is coupled to the tissue cutter by, e.g., a torque shaft that extends through the catheter tube. Operation of the motor 60 rotates the tissue cutting blades 54. When rotated and deployed into their radially extended, deployed condition within a tissue mass, the tissue cutting blades 54 cut away surrounding tissue to form an enlarged cavity 62 within the tissue mass that, in size, approximates the maximum diameter of the tissue cutting blades 54 when in their radially extended, deployed condition.

Representative techniques for manipulating the third instrumentation component 16 are shown in FIGS. 15A to 15E). In this arrangement, the directions 38 for using the third instrumentation component 16 include passing the flexible drilling unit 50 over the K-wire 44 through the slotted guide tube 30 and formed access channel 48 with the tissue cutting blades 54 in their collapsed, lay-flat, low profile condition (as FIG. 15A shows). Under radiologic or CT monitoring, the flexible drilling unit 50 is passed over the K-wire 44 in the path defined by the K-wire 44 in a cephalad direction through the cortical bone of the end plate of the S1 vertebra, and the adjoining disc. When a desired position within the disc space is reached, the instructions 38 for use include operating the motor 60 to rotate the cutting blades 54 and placing the tissue cutting blades 54 in their radially extended, deployed condition (se FIG. 15B shows). The flexible drilling unit 50 is advanced with the cutting blades 54 extended (see FIGS. 15C and 15D). As a result, the tissue cutting blades 54 cut away surrounding tissue to form the enlarged cavity 62 within the disc space, which involves not only the removal of the disc material itself, but also desirable involves the removal of a portion of the cortical bone bordering the disc material, as FIG. 15D shows. In a representative embodiment, the diameter of the cavity 62 is about 6 to 10 mm.

The instructions 38 for use include, after formation of the cavity 62, the return of the tissue cutting blades 54 to their collapsed, lay-flat low profile condition and the withdrawal of the flexible drilling unit 50 over the K-wire 44, as FIG. 15E shows. Remnants of cortical bone and/or disc tissue can be aspirated from the cavity 62 by suction, if required.

Percutaneous disc cavity creation in the space between the first targeted vertebral body (i.e., the S1 vertebra) and the second targeted vertebral body (i.e., the L5 vertebra) has been achieved, as shown in FIG. 15E.

D. Achieving Percutaneous Disc Cavity Support

1. Deployment of a Self-Expanding Support Matrix or Structure

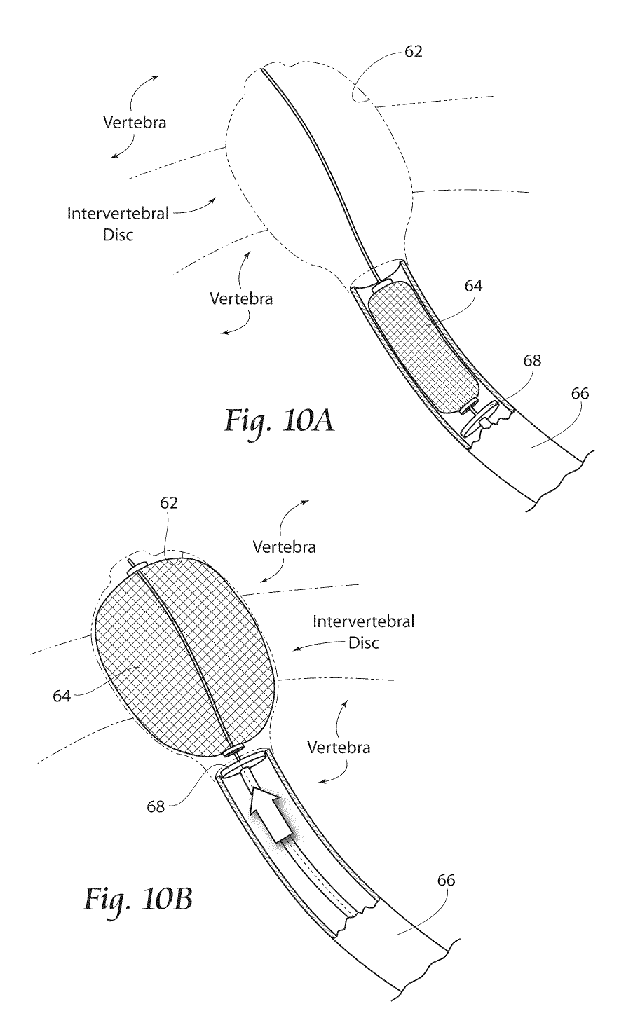

In one representative embodiment shown in FIG. 8E, the fourth instrumentation component 18 includes a self-expanding support matrix or structure 64 formed from a resilient metal or mesh fabric comprising a plurality of resilient strands of a resilient material that has been, e.g., heat treated to substantially set a desired shape. As further shown in FIG. 10A, the self-expanding support matrix or structure 64 can be collapsed and inserted into the lumen of a delivery catheter 66. The self-expanding support matrix or structure 64 is urged through the catheter 66 and out the distal end (see FIG. 10B), whereupon it will self-expand in situ to return to its expanded state at the targeted treatment site.

In this arrangement (see FIG. 8E and as further shown in FIGS. 10A and 10B), the fourth instrumentation component 18 also includes a flexible delivery catheter 66 with is sized and configured to be deployed over the K-wire 44. The delivery catheter 66 receives the self-expanding support matrix or structure 64 in its collapsed state (see FIG. 10A). A pusher 68 that slides within the catheter 66 advances the collapsed self-expanding support matrix or structure 64 out the distal end of the catheter 66 at the targeted treatment site (see FIG. 10B).

The self-expanding support matrix or structure 64 is sized and configured so that, when expanded within the enlarged cavity 62 formed by the third instrumentation component 16 (as previously described) (see, e.g., FIG. 16B), the self-expanding support matrix or structure 64 assumes a physical geometry and mechanical strength that restores the functionality of the intervertebral disc to eliminate the grating motion and nerve impingement between the adjacent vertebrae caused by disc/facet degeneration, increasing the space for the nerve roots, stabilize the spine, restore spine alignment, and relieve pain.

The self-expanding support matrix or structure 64 can be made of a biodegradable materials, e.g., polylactide (PLA), which is a biodegradable, thermoplastic, aliphatic polyester derived from renewable resources, such as corn starch or sugarcanes.

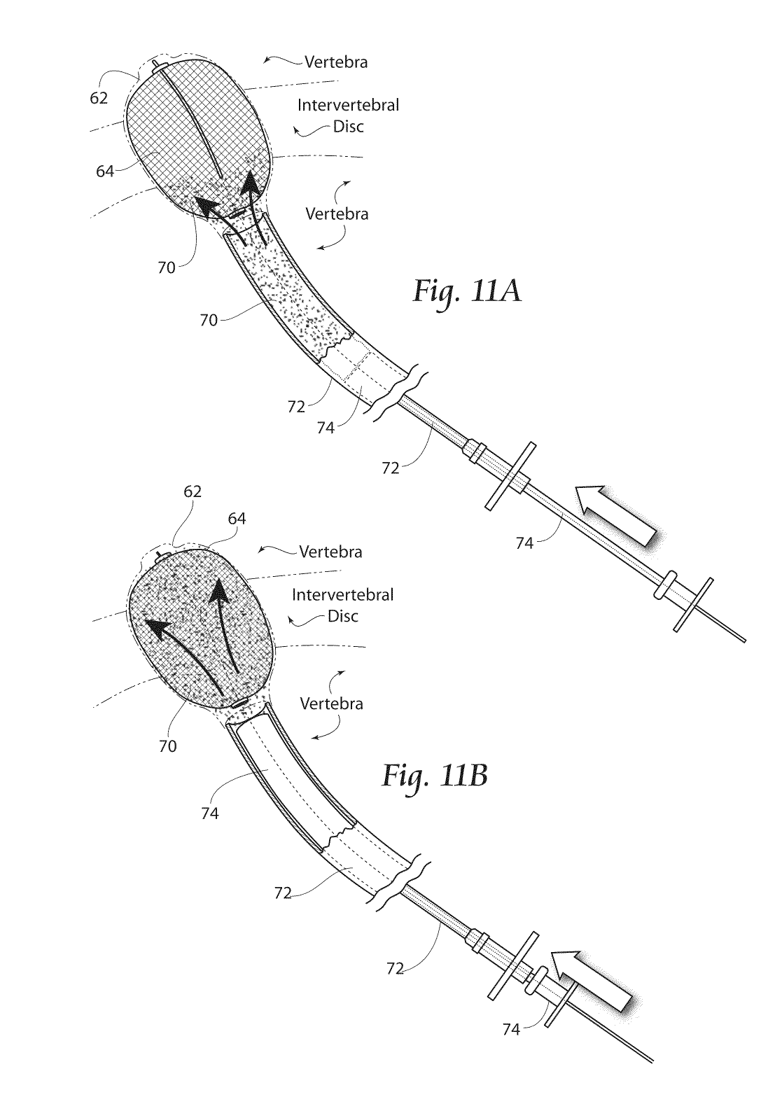

Desirably, the self-expanding support matrix or structure 64 is also sized and configured so that, when expanded, an interior chamber is formed that can accommodate a bone filling material 70, as shown in FIGS. 11A and 11B). The bone filling material 70 can comprise, e.g., a flowable polymer material can comprise, e.g., poly(methyl methacrylate) (PMMA), which is a transparent thermoplastic; or polylactic acid or polylactide (PLA). The bone filling material 70 can also comprise autologous or allograft bone graft material.

In this arrangement (see FIG. 8E and as further shown in FIGS. 11A and 11B), the fourth instrumentation component 18 also includes a flexible bone filling material delivery cannula 72 that is sized and configured to be deployed over the K-wire 44. The flexible bone filling material delivery cannula 72 conveys the bone filling material 70 or bone graft material into the interior chamber of the self-expanding support matrix or structure 64 after its expansion within the formed enlarged cavity 62. As FIGS. 11A and 11B show, a tamping tool 74 that slides within the flexible bone filling material delivery cannula 72 can be used to expel the bone filling material 70 or bone graft material out the distal end of the bone filling material delivery cannula 72, packing the material into the interior chamber.

Gaps between adjacent strands of the support matrix or structure 64 allow the bone filling or bone graft materials 70 introduced within the chamber to flow outside the support matrix or structure 64 and occupy space outside the support matrix or structure 64. Thus, bone filling material 70 or bone graft material packed into structure after its expansion within the formed enlarged cavity 62, begins to grow through the gaps eventually forming a solid bond or fusion holding the vertebrae together, forming a strong and stable construct.

Representative techniques for manipulating the fourth instrumentation component 18 are shown in FIGS. 16A to 16E). In this arrangement, the directions 38 for using the fourth instrumentation component 18 include collapsing and inserted the self-expanding support matrix or structure 64 into the lumen of the flexible delivery catheter 66. The instructions 38 include passing the flexible delivery catheter 66, under radiologic or CT monitoring, over the K-wire 44 to position the distal end of the catheter in the formed enlarged cavity 62, as FIG. 16A shows. As FIG. 16A further shows, the instructions 38 include manipulating the pusher 68 to urge the self-expanding support matrix or structure 64 through the catheter 66 and out the distal end. The support matrix or structure 64 will self-expand and return to its expanded state within the formed enlarged cavity 62, as FIG. 16B shows.

The instructions 38 further include withdrawing the delivery catheter 66 over the K-wire 44 (see FIG. 16B) and passing the flexible bone filling material delivery cannula 72 over the K-wire 44 into communication with the interior chamber of the support matrix or structure 64, now expanded within the cavity 62 (see FIG. 16C). The instructions 38 include manipulating the tamping tool 74 to expel bone filling material or bone graft material 70 out the distal end of the bone filling material delivery cannula 72, to pack the material into the interior chamber, as FIG. 16D shows.

Percutaneous disc cavity support has been achieved, as FIG. 16E shows.

2. Cast-In-Place Support Matrix or Structure

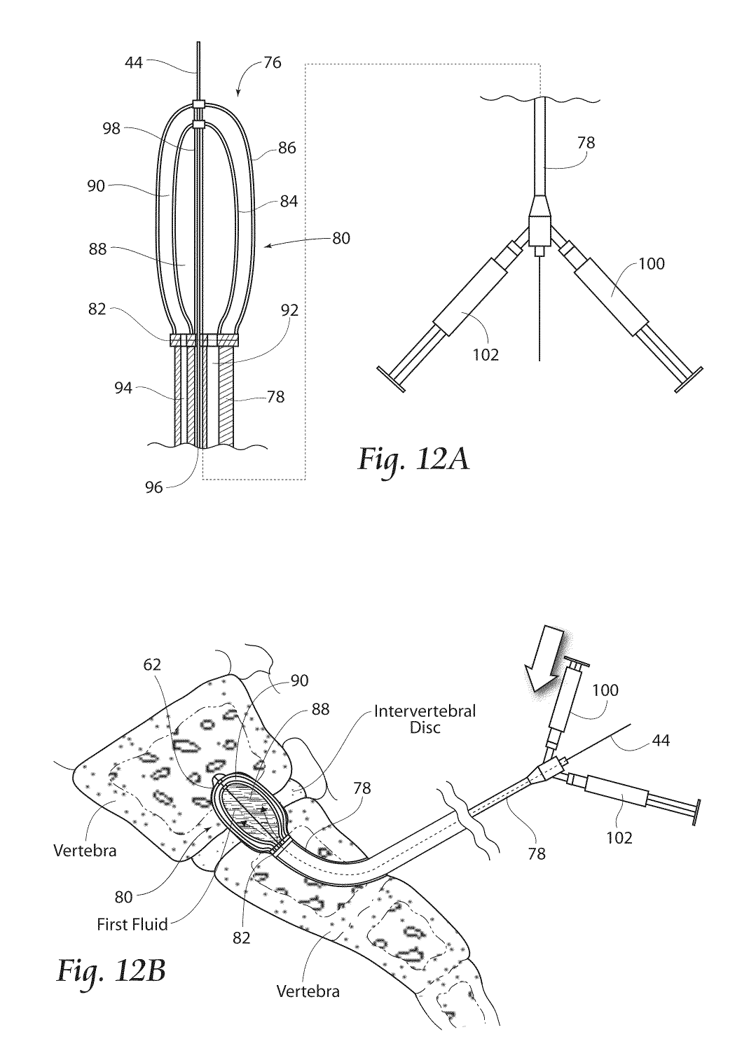

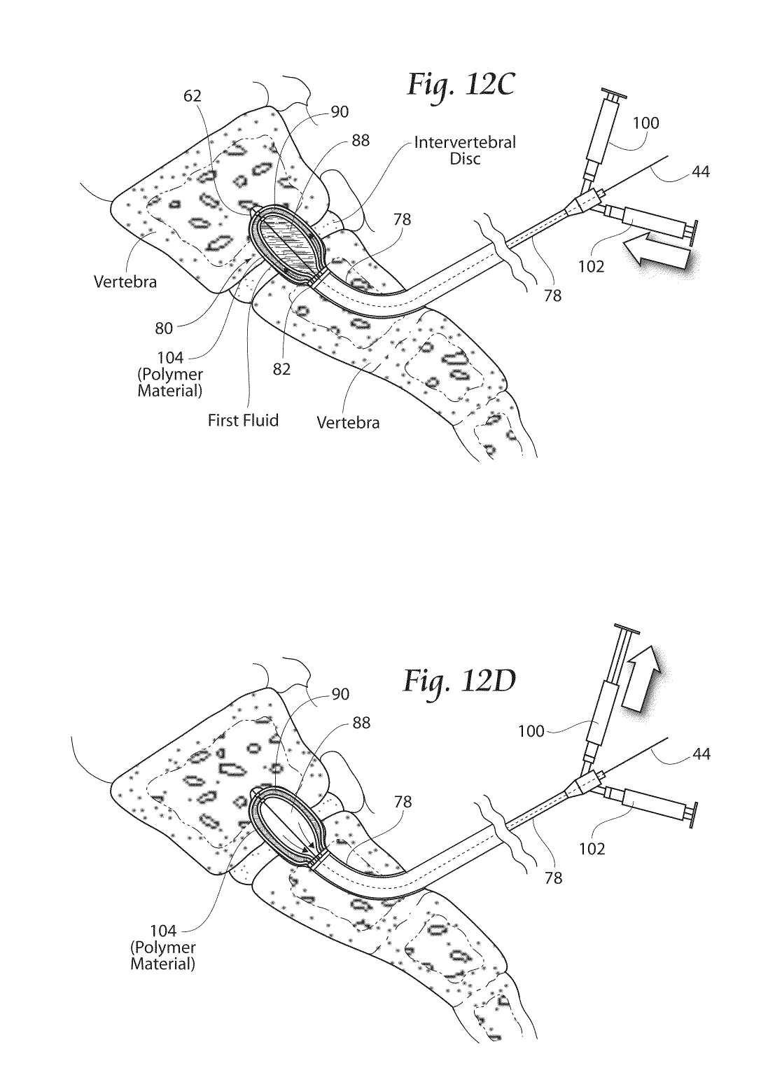

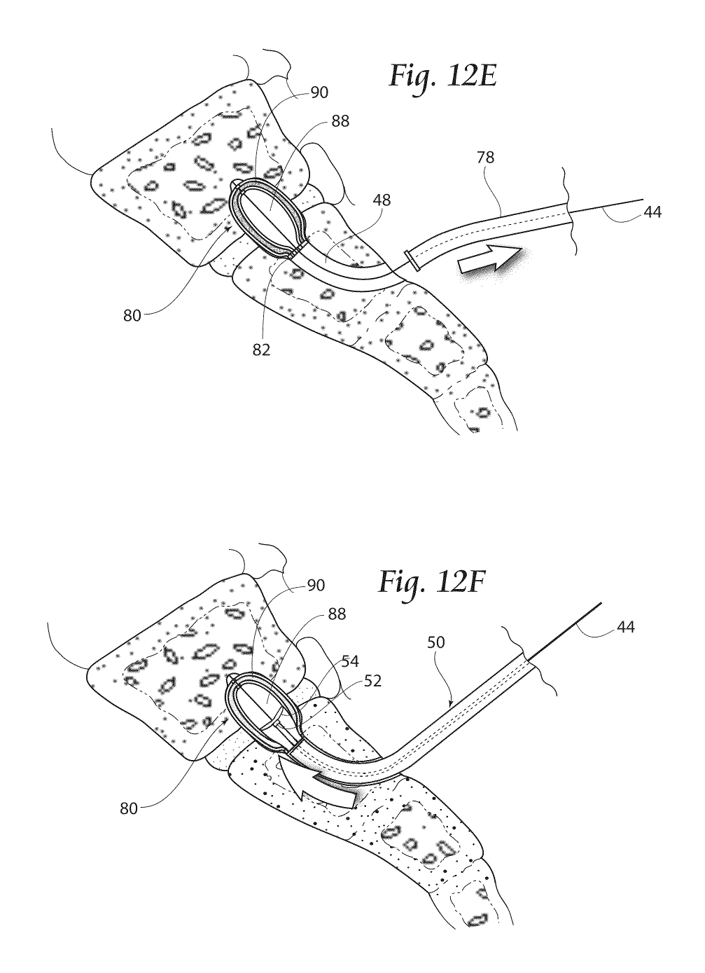

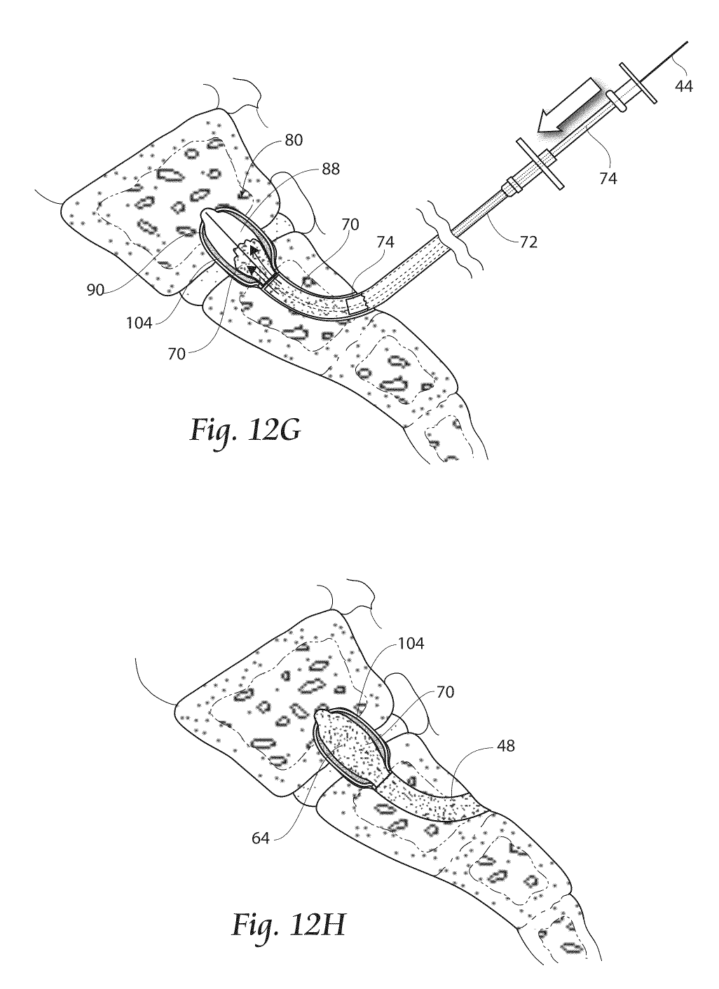

In another representative embodiment shown in FIGS. 12A to 12H, the fourth instrumentation component 18 includes an in-situ molding component 76 sized and configured to cast in place within the enlarged cavity 62 formed by the third instrumentation component 16 a polymeric support matrix or structure 64. Like the self-expanding support matrix or structure 64 previously described, the cast-in-place polymeric support matrix is sized and configured so that, after being cast in situ within the formed enlarged cavity 62 (see FIG. 12H), it presents a structure having the physical geometry and mechanical strength that restores the functionality of the intervertebral disc, to eliminate the grating motion and nerve impingement between the adjacent vertebrae caused by disc/facet degeneration, increasing the space for the nerve roots, stabilize the spine, restore spine alignment, and relieve pain.

The in-situ molding component 76 can be variously configured. In the representative embodiment shown in FIGS. 12A to 12H, the in-situ molding component 76 comprises a flexible catheter tube 78 carrying at its distal end a concentric expandable assembly 80 (see FIG. 12A). The distal end of the catheter tube includes a frangible connection 82 (shown in FIG. 12A), which permits the selective separation of the concentric expandable assembly 80 from the catheter tube 78 (see FIG. 12E), e.g., by rotation of the catheter tube 78 relative to the concentric expandable assembly 80.

As shown in FIG. 12A, the concentric expandable assembly 80 comprises a first elastic or semi-elastic wall 84 defining an inner expandable chamber 88. The inner expandable chamber 88 is concentrically surrounded by a second elastic or semi-elastic wall 86 defining an outer expandable chamber 90. The outer expandable chamber 90 occupies the space between the inner wall material 84 and the outer wall material 86. Due to their concentric nature, the size and geometry of the outer chamber 90 generally conforms to the size and geometry of the inner chamber 88.

Prior to separation of the concentric expandable assembly 80 from the catheter tube 78 (as shown in FIG. 12A), a first lumen 92 in the catheter tube 78 communicates with the inner expandable chamber 88 to convey fluid into the inner chamber 88. Prior to separation of the concentric expandable assembly 80 from the catheter tube 78, a second lumen 94 in the catheter tube 78 communicates with the outer expandable chamber 90 to convey fluid into the outer chamber 90, independent of fluid delivery through the first lumen 92. A third lumen 96 in the catheter tube 78 (which communicates with a thru-lumen 98 in the center of the concentric expandable assembly 80) allows passage of the K-wire 44 through the catheter tube 78 and concentric expandable assembly 80.

In this arrangement (shown in FIG. 12A), the in-situ molding component 76 further comprises a first pressurized source of fluid 100 coupled to the first lumen 92. Operation of the first pressurized source of fluid 100 introduces the first fluid into the inner chamber 88 (see FIG. 12B), to enlarge the outer diameter of the inner chamber 88. The first source of fluid 100 can comprise, e.g., a syringe containing saline, which is desirably mixed with a material that is visible to fluoroscopic visualization, such as iodine. Operation of the syringe 100 causes the fluid to fill and enlarge the inner chamber 88 under pressure, which can preferably be monitored by fluoroscopy. The outer chamber 90 likewise enlarges in geometry and outer diameter, conforming to the expansion of the inner chamber 88.

The in-situ molding component 76 comprises a second pressurized source of fluid 102 coupled to the second lumen 94 (shown in FIG. 12A). The second source of fluid 102 can comprise, e.g., a syringe containing a flowable polymer material 104 that sets to a hardened condition. The flowable polymer material 104 can comprise, e.g., poly(methyl methacrylate) (PMMA), which is a transparent thermoplastic; or polylactic acid or polylactide (PLA). Operation of the syringe 102 (see FIG. 12C) causes the fluid to fill and enlarge the outer chamber 90 under pressure. Over time, the flowable polymer material 104 sets to a hardened condition within the outer chamber 90, on forming to the geometry of the outer chamber 90, to form the polymeric support matrix 64 that has been cast-in-place (see FIG. 12E. The temperature of the fluid delivered to the inner chamber 88 can be controlled to provide desired glass transition temperature conditions to accelerate the in situ set-up of the polymer 104.

Representative techniques for manipulating the in-situ molding component 76 are shown in FIGS. 12B to 12H. In this arrangement, the directions 38 for using the in-situ molding component 76 include deploying the in-situ molding component 76 over the K-wire 44, under radiologic or CT monitoring, to position the concentric expandable assembly 80 in the formed enlarged cavity 62 (see FIG. 12B). As also shown in FIG. 12B, the instructions 38 include operating the first source of fluid 100 to introduce the first fluid under pressure into the inner chamber 88. The introduction of the first fluid expands and enlarges the diameter of the concentric expandable assembly 80 within the formed enlarged cavity 62, as FIG. 12B shows.

The expansion of the concentric expandable assembly 80 within the cavity 62 percutaneously formed in the intervertebral space, forces the vertebrae (S1-L5) apart, while also distracting and decompressing the nerve roots, stabilizing the spine, restoring spine alignment, and relieving pain.

The instructions 38 also include operating the second source of fluid 102, under radiologic or CT monitoring, to introduce the second (polymeric) fluid 104 under pressure into the outer chamber 90, as shown in FIG. 12C. As FIG. 12C shows, the flowable polymer material 104 enters the outer chamber 90, conforming to the favorable geometry established by the expansion of the first chamber 88. In the space defined between the inner and outer walls, the flowable polymer material 104 sets to a hardened condition to form in situ the polymeric support matrix 64. The space defined between the inner and outer walls serves as a mold in which the polymeric support matrix 64 has been cast-in-place in situ in the intervertebral disc space between the targeted first and second vertebral bodies.

The instructions 38 also include, after sufficient set up of the polymer support matrix, the separation of the catheter tube 78 from the concentric expandable assembly 80. Fluid resident in the inner chamber 88 is aspirated, to conflate the inner chamber 88 (as FIG. 12D shows). The presence of the polymer support matrix 64 will frictionally stabilize the position of the concentric expandable assembly 80 within the intervertebral disc space sufficient to allow the catheter tube 78 to be rotated relative to the concentric expandable assembly 80 (see FIG. 12E), thereby permitting separation of the catheter tube 78 from the concentric expandable assembly 80. The catheter tube 78 is withdrawn over the K-wire 44, as FIG. 12E shows, leaving the formed-in-place polymer support matrix 64 behind.

The instructions 38 can further include passing the flexible drilling unit 50 (the third instrumentation component 16) over the K-wire 44 through the slotted guide tube 30 with the tissue cutting blades 54 in their collapsed, lay-flat, low profile condition. Under radiologic or CT monitoring, the flexible drilling unit 50 is passed over the K-wire 44 in the path until a desired position near the periphery of the cast-in-place polymer support matrix 64 within the disc space is reached. The instructions 38 for use include operating the motor 60 to rotate the cutting blades 54 and placing the tissue cutting blades 54 in their radially extended, deployed condition to form an enlarged central lumen through the cast-in-place polymer support matrix 64 (see FIG. 12F). The instructions 38 further include withdrawing the flexible drilling unit 50 (the third instrumentation component 16) over the K-wire 44 and passing the flexible bone filling material delivery cannula 72 over the K-wire 44 into communication with the central lumen formed within the cast-in-place support matrix 64 (see FIG. 12G). The instructions 38 include manipulating the tamping tool 74 (see FIGS. 12G and 12H) to expel bone filling material or bone graft material 70 out the distal end of the bone filling material delivery cannula 72, to pack the material into the central lumen of the cast-in-place support matrix 64.

Percutaneous disc cavity support has been achieved. The K-wire 44 and slotted guide tube 30 are removed, as FIG. 12H shows).

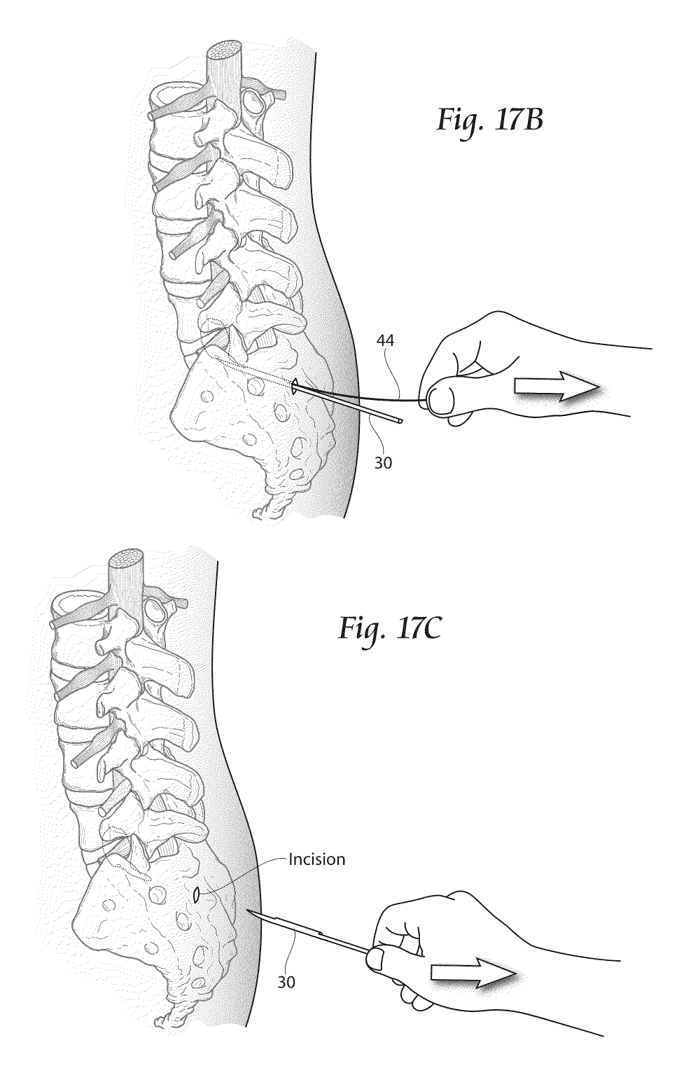

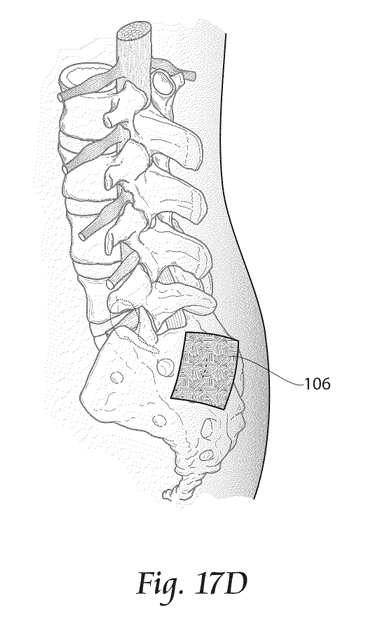

After percutaneous disc cavity support has been achieved, the K-wire 44 is removed (FIG. 17A), as is the slotted guide tube 30 (FIG. 17B). A bandage 106 is placed over the percutaneous access site (FIG. 17C).

E. Bilateral/Multi-Level Procedure

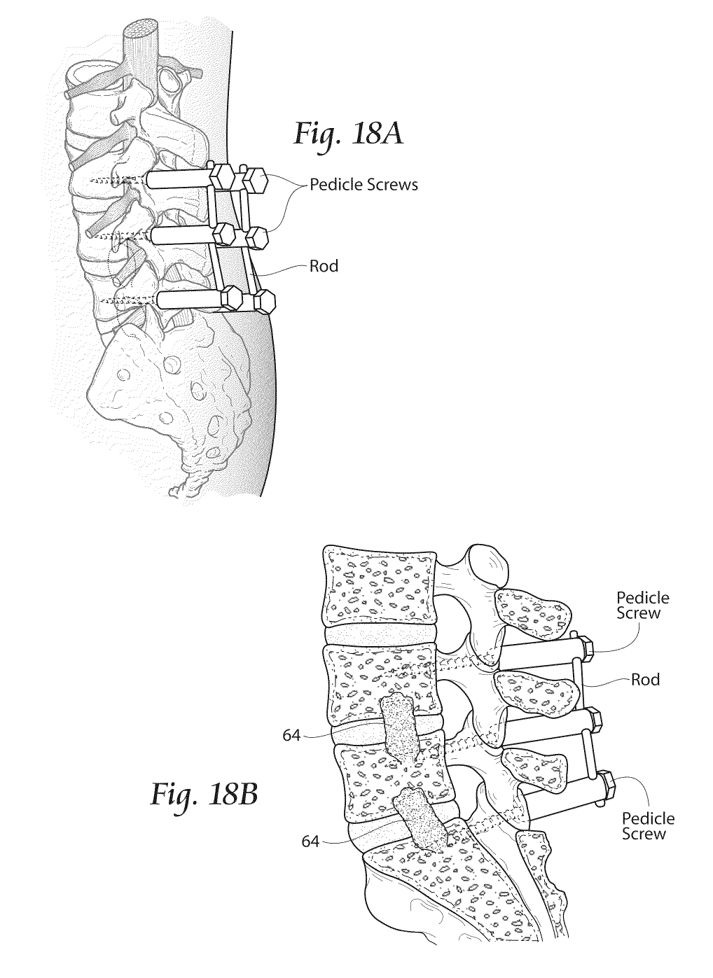

Both left and right sides and multiple levels can be treated during the same procedure using the instrumentation and the techniques described above. For example, FIG. 18B shows the results of a procedure treating three adjacent vertebral bodies S1, L5, and L4, each with percutaneous, bilateral transpedicular accesses. The multiple level bilateral procedure entails the bilateral, transpedicular deployment of six slotted guide tubes 30 and six K-wires 44, two in each vertebral body S1, L5, and L4. In this arrangement, the same second, third and fourth instrumentation components can be used in the manner described sequentially at each bilateral, transpedicular site.

F. Ancillary Use of Pedicle Screws, Plates, and Rods

As shown in FIGS. 18A and 18B, following use of the instrumentation and the techniques described above, pedicle screws, plates, and rods can be installed in conventional fashion to hold the targeted vertebrae in place while the spine fusion heals. Pedicle screws can be percutaneously placed through the pedicle bone on the back of the spinal column, one on each side of a targeted vertebra. The screws can be coupled to metal rods that connect all the screws together, to create a stiff metal frame that holds the vertebrae still so that healing can occur.

### III. Conclusion

The systems, devices, and surgical procedures described treat degenerative changes in the spine by performing arthrodesis between vertebrae in the spine using minimally invasive instrumentation and techniques. Such systems, devices, and surgical procedures make possible a minimally-invasive spine fusion procedure that would require less than a 24 hospital stay, provide maximum benefit for patient, minimize cost of hospitalization and infection, and minimize patient recovery and return to work.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A system for percutaneous fusion of the spine comprising
a first instrumentation component that is sized and configured to achieve posterior percutaneous transpedicular access to an interior of a first targeted vertebral body through a pedicle of the vertebra,
a second instrumentation component that is sized and configured to achieve percutaneous cephalad trans-disc access to an interior of a second targeted vertebral body at a next adjacent superior level to the first targeted vertebral body,
a third instrumentation component that is sized and configured to achieve percutaneous disc cavity creation comprising a device for forming an enlarged cavity in the intervertebral disc space between the first and second targeted vertebral bodies, and
a fourth instrumentation component that is sized and configured to achieve percutaneous disc cavity support comprising a support matrix placed in the enlarged cavity formed by the third instrumentation component and that is sized and configured to separate and hold the first and second vertebral bodies apart, to thereby distract nerve roots and relieve pressure on the nerves, and a device for conveying in a percutaneous manner a volume of a filling material into the support matrix that, over time, hardens to promote fusion of the targeted first and second vertebral bodies.

2. A system according to claim 1
wherein the filling material comprises a flowable polymer material or a bone graft material that, upon setting, helps the vertebrae heal together or fuse.

3. A system according to claim 1
wherein the first instrumentation component comprises a guide tube including a proximal slotted side wall and distal slotted side wall oriented, in use, to face in a caphalad direction.

4. A system according to claim 1
wherein the second instrumentation component comprises a stylus or suture instrument having a curvature that is sized and configured to be passed through a guide tube in a curvilinear path to direct a distal end of the stylus or suture instrument through the superior end plate of the first targeted vertebral body into and through the intervertebral disc between the first and second targeted vertebral bodies and into and through the inferior (caudal) end plate of the second targeted vertebral body.

5. A system according to claim 4
wherein the curvature of the stylus or suture instrument is pre-formed.

6. A system according to claim 4
wherein the curvature of the stylus or suture instrument is set at an instance of use.

7. A system according to claim 4
wherein the stylus or suture instrument includes a center lumen sized and configured to receive a curved guide wire, and
wherein the second instrumentation includes a cannulated drill that is sized and configured for passage over the guide wire to clear an access channel through cortical bone of the end plate of the first targeted vertebral body, into and through adjoining disc tissue, and into and through the end plate into cortical bone of the second targeted vertebral body.

8. A system according to claim 4
wherein the stylus or suture instrument includes a center lumen sized and configured to receive a curved guide wire to guide passage of the third instrumentation component.

9. A system according to claim 1
wherein the first instrumentation component comprises a guide tube including a proximal slotted side wall and distal slotted side wall oriented, in use, to face in a caphalad direction, and
wherein the second instrumentation component comprises a curved stylus or suture instrument that is sized and configured to be passed through the guide tube in a curvilinear path through the slotted proximal and distal side walls to direct the distal end of the stylus or suture instrument through the superior end plate of the first targeted vertebral body into and through the intervertebral disc between the first and second targeted vertebral bodies and into and through the inferior (caudal) end plate of the second targeted vertebral body.

10. A system according to claim 9
wherein the curvature of the stylus or suture instrument is pre-formed.

11. A system according to claim 9
wherein the curvature of the stylus or suture instrument is set at an instance of use.

12. A system according to claim 9
wherein the stylus or suture instrument includes a center lumen sized and configured to accommodate placement of a curved guide wire, and
wherein the second instrumentation includes a cannulated drill that is sized and configured for passage over the guide wire to clear an access channel through cortical bone of the end plate of the first targeted vertebral body, into and through adjoining disc tissue, and into and through the end plate into cortical bone of the second targeted vertebral body.

13. A system according to claim 9
wherein the stylus or suture instrument includes a center lumen sized and configured to receive a curved guide wire to guide passage of the third instrumentation component.

14. A system according to claim 1
wherein the third instrumentation component comprises a flexible tissue drilling unit including, at a distal end, a tissue cutter that is sized and configured to assume a collapsed, lay-flat low profile condition for passage through a guide tube and to be expanded toward a radially enlarged deployed condition for cutting tissue and forming an enlarged cavity in the intervertebral disc space between the first and second targeted vertebral bodies.

15. A system according to claim 14
wherein the first instrumentation component comprises a guide tube including a proximal slotted side wall and distal slotted side wall oriented, in use, to face in a caphalad direction, and
wherein the second instrumentation component comprises a curved stylus or suture instrument that is sized and configured to be passed through the guide tube in a curvilinear path through the slotted proximal and distal side walls to direct the distal end of the stylus or suture instrument through the superior end plate of the first targeted vertebral body into and through the intervertebral disc between the first and second targeted vertebral bodies and into and through the inferior (caudal) end plate of the second targeted vertebral body, the stylus or suture instrument including a center lumen sized and configured to accommodate placement of a curved guide wire to guide passage of the flexible tissue drilling unit during use.

16. A system according to claim 1
wherein the fourth instrumentation component includes a self-expanding support matrix or structure that is sized and configured so that, when expanded within the enlarged cavity, an interior chamber is formed that can accommodate the filling material.

17. A system according to claim 16
wherein the fourth instrumentation includes a flexible bone filling material delivery cannula that is sized and configured conveys the filling material into the interior chamber of the self-expanding support matrix or structure after its expansion within the enlarged cavity.

18. A system according to claim 16
wherein the first instrumentation component comprises a guide tube including a proximal slotted side wall and distal slotted side wall oriented, in use, to face in a caphalad direction, and
wherein the second instrumentation component comprises a curved stylus or suture instrument that is sized and configured to be passed through the guide tube in a curvilinear path through the slotted proximal and distal side walls to direct the distal end of the stylus or suture instrument through the superior end plate of the first targeted vertebral body into and through the intervertebral disc between the first and second targeted vertebral bodies and into and through the inferior (caudal) end plate of the second targeted vertebral body, the stylus or suture instrument including a center lumen sized and configured to accommodate placement of a curved guide wire to guide delivery of the self-expanding support matrix or structure.

19. A system according to claim 1
wherein the fourth instrumentation component includes an in-situ molding component that is sized and configured to cast in place within the enlarged cavity formed by the third instrumentation component a polymeric support matrix or structure that is sized and configured so that, after being cast, presents a structure having the physical geometry and mechanical strength that restores the functionality of the intervertebral disc.

20. A system according to claim 19
further including a flexible tissue drilling unit to form an enlarged central lumen through the cast-in-place polymer support matrix that is sized and configured to receive the filling material.

21. A system according to claim 19
wherein the first instrumentation component comprises a guide tube including a proximal slotted side wall and distal slotted side wall oriented, in use, to face in a caphalad direction, and
wherein the second instrumentation component comprises a curved stylus or suture instrument that is sized and configured to be passed through the guide tube in a curvilinear path through the slotted proximal and distal side walls to direct the distal end of the stylus or suture instrument through the superior end plate of the first targeted vertebral body into and through the intervertebral disc between the first and second targeted vertebral bodies and into and through the inferior (caudal) end plate of the second targeted vertebral body, the stylus or suture instrument including a center lumen sized and configured to accommodate placement of a curved guide wire to guide delivery of the fourth instrumentation component.

22. A system according to claim 1
wherein the first instrumentation component comprises a guide tube including a proximal slotted side wall and distal slotted side wall oriented, in use, to face in a caphalad direction, and
wherein the second instrumentation component comprises a curved stylus or suture instrument that is sized and configured to be passed through the guide tube in a curvilinear path through the slotted proximal and distal side walls to direct the distal end of the stylus or suture instrument through the superior end plate of the first targeted vertebral body into and through the intervertebral disc between the first and second targeted vertebral bodies and into and through the inferior (caudal) end plate of the second targeted vertebral body, the stylus or suture instrument including a center lumen sized and configured to accommodate placement of a curved guide wire to guide subsequent delivery of the third and fourth instrumentation components.

23. A method for percutaneous fusion of the spine comprising
(i) percutaneously manipulating instrumentation to achieve posterior percutaneous transpedicular access to an interior of a first targeted vertebral body through a pedicle of the vertebra,
(ii) percutaneously manipulating instrumentation through the percutaneous transpedicular access achieved during (i), to achieve percutaneous cephalad trans-disc access to an interior of a second targeted vertebral body at a next adjacent superior level to the first targeted vertebral body,
(iii) percutaneously manipulating instrumentation through the percutaneous transpedicular access achieved during (i) and the percutaneous cephalad trans-disc access achieved during (ii), to achieve percutaneous disc cavity creation comprising forming an enlarged cavity in the intervertebral disc space between the first and second targeted vertebral bodies, and
(iv) percutaneously manipulating instrumentation through the percutaneous transpedicular access achieved during (i) and the percutaneous cephalad trans-disc access achieved during (ii), to achieve percutaneous disc cavity support comprising placing a support matrix in the enlarged cavity formed during (iii) that is sized and configured to separate and hold the first and second vertebral bodies apart, to thereby distract nerve roots and relieve pressure on the nerves, and conveying a volume of a filling material into the support matrix that, over time, hardens to promote fusion of the targeted first and second vertebral bodies.

24. A method according to claim 23
wherein (i) includes placing a guide tube including a proximal slotted side wall and distal slotted side wall oriented, in when placed, to face in a caphalad direction, and
wherein (ii) includes placing a curved stylus or suture instrument through the guide tube in a curvilinear path through the slotted proximal and distal side walls to direct the distal end of the stylus or suture instrument through the superior end plate of the first targeted vertebral body into and through the intervertebral disc between the first and second targeted vertebral bodies and into and through the inferior (caudal) end plate of the second targeted vertebral body, and placing a curved guide wire through the guide tube to guide subsequent percutaneous manipulation of instrumentation during (iii) and (iv).

25. A method according to claim 24
wherein (iii) includes manipulating a flexible tissue drilling unit including, at a distal end, a tissue cutter that is sized and configured to assume a collapsed, lay-flat low profile condition for passage over the guide wire and to be expanded toward a radially enlarged deployed condition for cutting tissue and forming an enlarged cavity in the intervertebral disc space between the first and second targeted vertebral bodies.

26. A method according to claim 24
wherein (iv) includes manipulating a self-expanding support matrix or structure that is sized and configured so that, when expanded within the enlarged cavity formed during (iii), an interior chamber is formed that can accommodate the filling material.

27. A method according to claim 26
wherein (iv) includes manipulating a flexible bone filling material delivery cannula to convey the filling material into the interior chamber of the self-expanding support matrix or structure after its expansion within the enlarged cavity.

28. A method according to claim 24
wherein (iv) includes manipulating an in-situ molding component to cast in place within the enlarged cavity formed during (iii) a polymeric support matrix or structure that, after being cast, presents a structure having the physical geometry and mechanical strength that restores the functionality of the intervertebral disc, and forming an enlarged central lumen through the cast-in-place polymer support matrix that is sized and configured to receive the filling material.

29. A system for percutaneous lumbar fusion comprising
a first instrumentation component that is sized and configured to achieve posterior percutaneous transpedicular access to an interior of a first targeted vertebral body through a pedicle of the vertebra,
a second instrumentation component that is sized and configured to achieve percutaneous cephalad trans-disc access to an interior of a second targeted vertebral body at a next adjacent superior level to the first targeted vertebral body,
a third instrumentation component that is sized and configured to achieve percutaneous disc cavity creation comprising a device for forming an enlarged cavity in the intervertebral disc space between the first and second targeted vertebral bodies,
a fourth instrumentation component that is sized and configured to achieve percutaneous disc cavity support comprising a support matrix placed in the enlarged cavity formed by the third instrumentation component and that is sized and configured to separate and hold the first and second vertebral bodies apart, to thereby distract nerve roots and relieve pressure on the nerves, and a device for conveying in a percutaneous manner a volume of a filling material into the support matrix that, over time, hardens to promote fusion of the targeted first and second vertebral bodies, and
instructions for manipulating the first, second, third, and fourth instrumentation components comprising
(i) percutaneously manipulating the first instrumentation component to achieve posterior percutaneous transpedicular access to an interior of a first targeted vertebral body through a pedicle of the vertebra,
(ii) percutaneously manipulating the second instrumentation component through the percutaneous transpedicular access achieved during (i), to achieve percutaneous cephalad trans-disc access to an interior of a second targeted vertebral body at a next adjacent superior level to the first targeted vertebral body,
(iii) percutaneously manipulating the third instrumentation component through the percutaneous transpedicular access achieved during (i) and the percutaneous cephalad trans-disc access achieved during (ii), to achieve percutaneous disc cavity creation comprising forming an enlarged cavity in the intervertebral disc space between the first and second targeted vertebral bodies, and
(iv) percutaneously manipulating the fourth instrumentation component through the percutaneous transpedicular access achieved during (i) and the percutaneous cephalad trans-disc access achieved during (ii), to achieve percutaneous disc cavity support comprising placing a support matrix in the enlarged cavity formed during (iii) that is sized and configured to separate and hold the first and second vertebral bodies apart, to thereby distract nerve roots and relieve pressure on the nerves, and conveying a volume of a filling material into the support matrix that, over time, hardens to promote fusion of the targeted first and second vertebral bodies.

30. A system according to claim 29
wherein the instructions for use (i) includes placing a guide tube including a proximal slotted side wall and distal slotted side wall oriented, in when placed, to face in a caphalad direction, and
wherein the instructions for use (ii) includes placing a curved stylus or suture instrument through the guide tube in a curvilinear path through the slotted proximal and distal side walls to direct the distal end of the stylus or suture instrument through the superior end plate of the first targeted vertebral body into and through the intervertebral disc between the first and second targeted vertebral bodies and into and through the inferior (caudal) end plate of the second targeted vertebral body, and placing a curved guide wire through the guide tube to guide subsequent percutaneous manipulation of the third and instrumentation components during (iii) and (iv).
